(19) United States
(12) Patent Application Publication
(10) Pub. No.: US 2026/0363568 A1
(43) Pub. Date: Nov. 19, 2026

(54) EVALUATION APPARATUS

(71) Applicant: Asahi Kasei Microdevices Corporation, Tokyo (JP)

(72) Inventors: Kengo HIROMOTO, Tokyo (JP); Takeji FUJIBAYASHI, Tokyo (JP)

(21) Appl. No.: 19/672,742

(22) Filed: May 11, 2026

(30) Foreign Application Priority Data
May 16, 2025 (JP) 2025-082405
Mar. 19, 2026 (JP) 2026-045437

(51) Int. Cl. A61B 5/05; A61B 5/00; A61B 5/11; G01S 13/08; G01S 13/88

(52) U.S. Cl. CPC A61B 5/05; A61B 5/1116; A61B 5/4205; A61B 5/4806; A61B 5/6892; A61B 5/7282; G01S 13/08; G01S 13/88; A61B 2503/08

(57) ABSTRACT

Provided is an evaluation apparatus including: a signal acquisition unit which acquires a signal indicating a temporal change in a distance from a millimeter-wave radar to a body surface of a subject, the signal being obtained by the millimeter-wave radar receiving reflected waves of millimeter waves radiated from the millimeter-wave radar to the body surface; and an evaluation unit which evaluates at least one of a swallowing function or an aspiration state of the subject based on the temporal change in the distance indicated by the signal. The evaluation unit may identify, in a waveform indicating the temporal change in the distance, a plurality of peaks indicating changes in distance exceeding a predetermined first threshold included within a predetermined period, and the evaluation unit may evaluate the swallowing function based on each interval of the plurality of peaks.

The contents of the following patent application(s) are incorporated herein by reference:

NO. 2025-082405 filed in JP on May 16, 2025

NO. 2026-045437 filed in JP on March 19, 2026

### BACKGROUND

### 1. Technical Field

The present invention relates to an evaluation apparatus.

### 2. Related Art

Patent Document 1 describes a “system for evaluating an eating and swallowing function of an evaluation subject by analyzing a voice of the evaluation subject”. Patent Document 2 describes “a swallowing function analysis system and a program for detecting and analyzing a sound including a vibration generated from a neck of a user in association with a swallowing action”. Patent Document 3 describes an “analysis system that enables screening for aspiration in which food or drink enters a trachea instead of an esophagus, based on characteristics of a swallowing sound of a patient”.

### RELATED ART DOCUMENTS

### Patent Documents

Patent Document 1: International Publication No. 2019/225242

Patent Document 2: Japanese Patent Application Publication No. 2019-051129

Patent Document 3: Japanese Patent Publication No. 4011071

### BRIEF DESCRIPTION OF THE DRAWINGS

FIG. 1 is a conceptual view illustrating an example of an installation example of an evaluation system 100.

FIG. 2 is a conceptual view illustrating an example of the installation example of the evaluation system 100.

FIG. 3 is a block diagram illustrating an example of a configuration of the evaluation system 100.

FIG. 4 is a diagram for describing an example of a detailed configuration of some blocks in the evaluation system 100 illustrated in FIG. 3.

FIG. 5 illustrates an example of a waveform 80 acquired using millimeter waves and a waveform 82 of a signal obtained by processing the waveform 80.

FIG. 6 is an example of the waveform 82 indicating swallowing.

FIG. 7 is an example of a waveform pattern indicating an abnormality in evaluation of presence or absence of an aspiration state.

FIG. 8 is an example of a waveform pattern indicating a normal state in the evaluation of the presence or absence of the aspiration state.

FIG. 9 is an example of an enlarged view obtained by stretching FIG. 7 in a time direction with a region D as a center.

FIG. 10 is an example of an enlarged view obtained by stretching FIG. 8 with a region E and a region F as centers.

FIG. 11 is an example of a flowchart for describing an evaluation method for the aspiration state and a swallowing function according to an embodiment.

FIG. 12 is a flowchart illustrating an example of processing for posture information generation performed by a posture acquisition unit 30.

FIG. 13 illustrates an example of a computer 1200 capable of embodying aspects of the present embodiment in whole or in part.

### DESCRIPTION OF EXEMPLARY EMBODIMENTS

The following embodiments do not limit the invention according to the claims. In addition, not all of the combinations of features described in the embodiments are essential to the solving means of the invention.

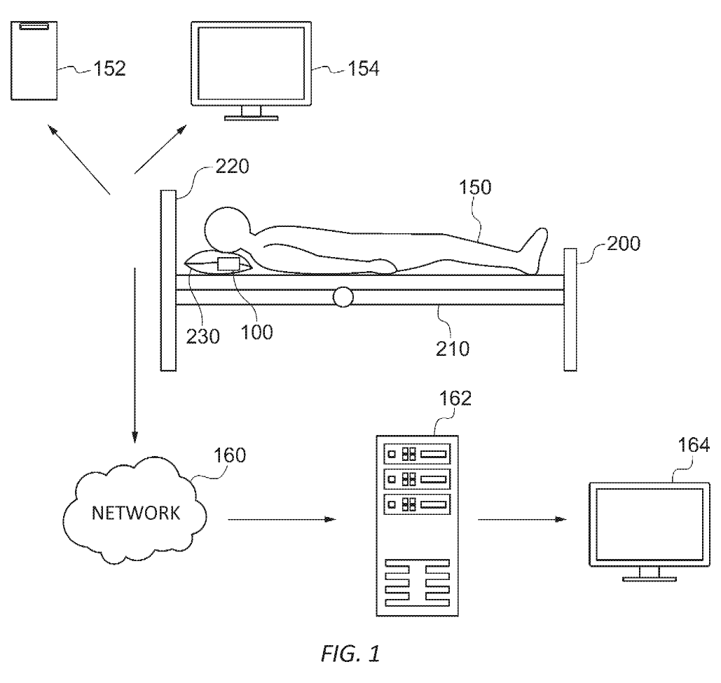

FIG. 1 is a conceptual view illustrating an example of an installation example of an evaluation system 100. The evaluation system 100 is provided on a bed 200 in a nursing care facility, a hospital, a home, or the like. The evaluation system 100 is a system that evaluates an aspiration state and a swallowing function of a subject 150 by using millimeter waves.

Aspiration means that food or saliva or the like enters an airway instead of an esophagus. When aspiration occurs in an elderly person or the like, the elderly person or the like may develop aspiration pneumonia caused by, for example, inflow of bacteria into an airway, accompanying the aspiration, and chemical inflammation or the like due to inflow of acidic gastric acid and stomach contents into the airway. The aspiration pneumonia ranks high among causes of death of elderly persons in recent years. Therefore, in a nursing care facility or the like in which elderly persons who may have a risk of a decline in swallowing ability due to aging or the like reside, there is a demand for monitoring whether or not a care recipient has an aspiration state or monitoring a swallowing function of the care recipient.

Millimeter waves are electromagnetic waves having a frequency band of about 30 GHz or higher. The evaluation system 100 of the present embodiment may use electromagnetic waves in a band of about 30 GHz to about 300 GHz. Corresponding wavelengths are about 1 mm to about 10 mm, and have strong directivity. In particular, the evaluation system 100 may use a millimeter-wave radar that radiates millimeter waves of a frequency-modulated continuous wave (FMCW) method.

The evaluation system 100 irradiates the subject 150 with millimeter waves and detects a vibration of a body surface of a neck by measuring a signal based on reflected waves from the body surface of the neck of the subject 150. Thus, the vibration of the body surface generated when the subject 150 performs swallowing can be detected, and the vibration of the body surface accompanying the swallowing of the subject 150 can be detected by using a millimeter-wave output antenna and a millimeter-wave input antenna that can be installed farther away than a wave source and a detection apparatus in sound waves. The vibration of the body surface accompanying the swallowing of the subject 150 can be said to be a vibration of the body surface corresponding to swallowing sound. When the swallowing sound is generated, the vibration corresponding to the swallowing sound is also generated on the body surface of the neck of the subject 150. This vibration occurs as a temporal change in a distance to the neck. In the evaluation system 100, the temporal change in the distance to the neck of the subject 150 is measured by using millimeter waves, whereby the swallowing of the subject 150 can be evaluated by the evaluation system disposed remotely with a non-contact configuration.

Note that in the above, the evaluation system 100 detects, as an example, the vibration of the body surface generated in the neck, in accordance with the swallowing sound. However, the body surface on which the evaluation system 100 detects the vibration is not limited to the neck, and is only required to be a body surface of a site where a vibration generated in accordance with the swallowing sound can be detected. For example, a vibration appearing on a body surface other than the neck, such as a head, a back, or a chest, as a result of the swallowing sound being transmitted through a body may be measured by millimeter waves. Hereinafter, the description of the embodiment using the neck as an example is merely illustrative of an observation point of the vibration, and does not limit the observation point of the vibration to the neck, and the observation point of the vibration may be the body surface of the head, the back, or the chest, or the like.

In the evaluation system 100, the millimeter-wave radar that radiates millimeter waves may be covered with a non-metal cover. The millimeter waves have strong directivity, and a certain non-metal material is transparent to them. The non-metal material may be provided in a cleanable member.

The bed 200 is a bed used in a nursing care facility. The bed 200 may be, as an example, a nursing care bed. The bed 200 includes a bed base 210, a headboard 220, and bedding 230.

The bed base 210 is a base member of the bed 200 for the subject 150 to lie on. When the bed 200 is the nursing care bed, the bed 200 may be a movable bed, and may be a bed in which a portion corresponding to an upper body of the subject 150 rises when the subject 150 sits up, or at a time of meals, or the like. Thus, the subject 150 may eat a meal on the bed 200, and the evaluation system 100 may evaluate swallowing when the subject 150 eats the meal on the bed 200.

The headboard 220 is a board that is on a head side of the subject 150 when the subject 150 lies on the bed 200. The headboard 220 may have a structure extending from one side of the bed base 210.

The evaluation system 100 can be provided on either one of the bed base 210 or the headboard 220. In particular, in the drawing, the evaluation system 100 is provided in the bedding 230 disposed on the bed base 210. By providing the evaluation system 100 in the bedding 230, the millimeter-wave radar of the evaluation system 100 can be disposed within a predetermined distance range with respect to the neck of the subject 150.

Either one of the bed base 210 or the headboard 220 may include a fixture for fixing the evaluation system 100 to the bed 200 near the head of the subject 150. Further, the evaluation system 100 may be retrofittable to the bed 200. The evaluation system 100 may have a structure attachable to and detachable from the bed 200, and may be configured as a portable system. In particular, in this case, the evaluation system 100 may be configured to be battery-driven.

A user terminal 152 receives and displays an evaluation result by the evaluation system 100. The evaluation result by the evaluation system 100 may be data-linked with a health management application operating on the user terminal 152. A monitor 154 may be a display unit 40 described later, which is a part of the evaluation system 100.

Further, when it is detected that the subject 150 is in an aspiration state or that the swallowing function has declined, information indicating the evaluation result may be transmitted via the network 160 to a server 162 disposed in a medical facility or the like. In this case, in communication with the network 160, the evaluation system 100 has a function of encrypting personal information of the subject 150, thereby protecting privacy of the subject 150.

In the server 162, the evaluation result by the evaluation system 100 may be recorded together with an electronic medical record or a medical record. In this case, the evaluation result by the evaluation system 100 can be linked with medication information regarding the subject 150. Thus, for example, for the subject 150 evaluated by the evaluation system 100 as having a declined swallowing function and a high aspiration risk, a medicament to be administered can be provided in a form that is easy to swallow, such as jelly, instead of a tablet, thereby preventing aspiration.

Further, the evaluation result by the evaluation system 100 may be linked, via the network 160, with data for meal provision for the subject 150. Thus, for example, for the subject 150 evaluated by the evaluation system 100 as having a declined swallowing function and a high aspiration risk, aspiration can be prevented by taking measures, for example, adjusting the form of meals, such as making provided meals soft, or extending a mealtime.

Furthermore, the evaluation result by the evaluation system 100 may be linked, via the network 160, with a history and a schedule of swallowing training for the subject 150. Thus, for example, for the subject 150 evaluated by the evaluation system 100 as having a declined swallowing function and a high aspiration risk, it is possible to increase a frequency of swallowing training and provide feedback so as to maintain and improve the swallowing function.

Further, data stored in the server 162 may be displayed on the monitor 164 for review by medical personnel such as a doctor. Thus, it becomes possible for the medical personnel to perform appropriate medical treatment on the subject 150 determined by the evaluation system 100 to be in an aspiration state.

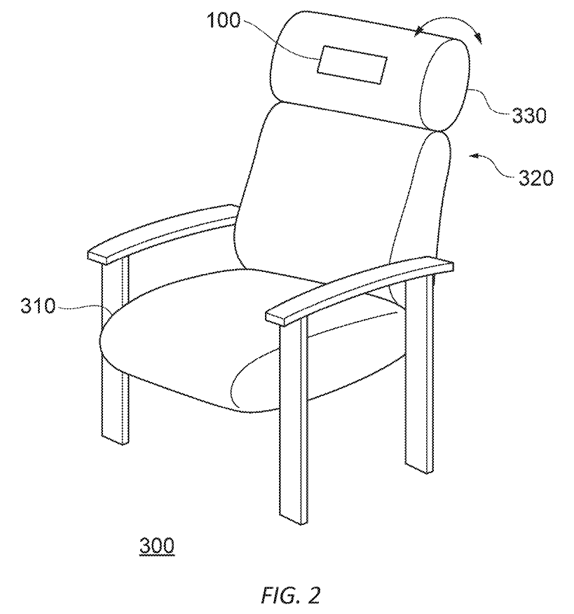

FIG. 2 is a conceptual view illustrating an example of the installation example of the evaluation system 100. A chair 300 may be a chair used at a time of meals in a nursing care facility.

The chair 300 is, as an example, a high-seat chair. However, a type of the chair 300 is not limited to the high-seat chair, and may be any type of chair used by the subject 150 for whom an aspiration state or a swallowing function is to be evaluated. The chair 300 of the present embodiment includes a seat surface 310 and a back surface 320.

The chair 300 may be a smart nursing care chair. The smart nursing care chair has a communication function in the nursing care chair itself, and can detect start of a meal of the subject 150 based on seating of the subject 150 on the chair 300, and seating of the subject 150 at a time close to a scheduled mealtime, and the like. The evaluation system 100 may operate in conjunction with the smart nursing care chair.

The seat surface 310 is a member for the subject 150 to sit on. The seat surface 310 may be provided at an appropriate hardness and height when used in a nursing care facility.

The back surface 320 is a member extending from one side of the seat surface, and is a member for supporting the back and head of the subject 150. The back surface 320 includes a support portion 330.

The support portion 330 is a member that supports the evaluation system 100. As an example, the support portion 330 may be a headrest of the chair 300. The support portion 330 supports the evaluation system 100. The support portion 330 that supports the millimeter-wave radar of the evaluation system 100 is included so as to be located within a predetermined range from the neck of the subject 150. The support portion 330 of the present embodiment has a structure whose angle is adjustable.

The evaluation system 100 may be provided inside a non-metal material of the support portion 330. The evaluation system 100 may be covered with a non-metal cover, and may be provided with a cleanable member. Further, the evaluation system 100 may be retrofittable to the chair 300. The evaluation system 100 may have a structure attachable to and detachable from the chair 300, and may be configured as a portable system. In particular, in this case, the evaluation system 100 may be configured to be battery-driven.

The evaluation system 100 may be provided by an "evaluation system installation member" having the support portion 330 that is a movable headrest of such a chair 300 or the like. However, the "evaluation system installation member" is not limited to the chair 300, and may be any installation member as long as it is an object on which the evaluation system can be installed and fixed, such as a washstand, a desk, or a table. Furthermore, the support portion of the installation member may be a support portion that supports only a millimeter-wave radar 10 that transmits and receives millimeter waves, rather than a support portion, such as the support portion 330, that supports the entire evaluation system 100 including the millimeter-wave radar 10 described later.

The support portion may be a suspension member that suspends the millimeter-wave radar 10 from a ceiling or a wall or the like by an arm-shaped base material, or may be a stand for fixing and installing the millimeter-wave radar 10 on a tabletop. Further, the support portion may be a support portion that is fixed to an installation target when supporting at least a part of the evaluation system 100 including the millimeter-wave radar 10. As described above, the support portion may be a support portion of any fixing method as long as it is a support portion that supports, on the installation target, the millimeter-wave radar 10 or at least a part of the evaluation system 100 when the subject 150 is irradiated with millimeter waves.

Although illustration is omitted in FIG. 2, the evaluation system 100 in FIG. 2 may also be connected to the user terminal 152 and the monitor 154, similarly to the evaluation system 100 illustrated in FIG. 1. Further, the evaluation system 100 may be connected to the server 162 via the network 160.

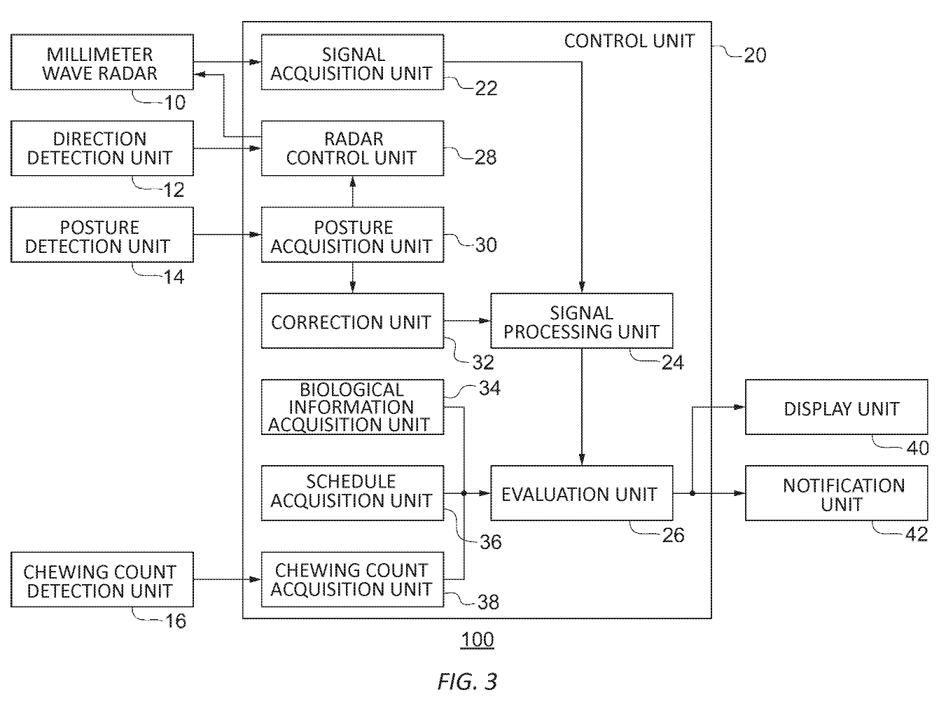

FIG. 3 is the block diagram illustrating an example of a configuration of the evaluation system 100. The evaluation system 100 includes the millimeter-wave radar 10, a direction detection unit 12, a posture detection unit 14, a chewing count detection unit 16, a control unit 20, the display unit 40, and a notification unit 42.

The evaluation system 100 evaluates the aspiration state and the swallowing function by irradiating the subject 150 with millimeter waves. The evaluation system 100 may be installed, for example, within 1.0 m from the neck of the subject 150, may be installed within a range within 40 cm, may be installed within a range within 20 cm, or may be installed within a range within 10 cm.

The evaluation system 100 may detect a change in distance of a predetermined point on the neck by irradiating the neck of the subject 150 with millimeter waves from behind the neck, and may detect a change in distance of a predetermined point on the neck by irradiating the neck with millimeter waves from a side of the neck. Further, the evaluation system 100 may detect a change in distance of a predetermined point on the neck by irradiating the neck with millimeter waves from a front of the neck. Further, a target which the evaluation system 100 irradiates with millimeter waves is not limited to the neck. The evaluation system 100 may detect a change in distance between the millimeter-wave radar 10 and a predetermined point by irradiating the body surface of the head, the back, the chest, or the like of the subject 150 with millimeter waves from the front or the rear. In any case, the swallowing sound can be acquired, but the evaluation system 100 can acquire stable swallowing sound data particularly when millimeter waves are radiated from behind the neck.

The millimeter-wave radar 10 includes an antenna that transmits and receives millimeter waves to and from the subject 150. A distance, an angle, and a velocity of a measurement point on the subject 150 can be measured based on relative angles of the measurement point, a transmitting antenna, and a receiving antenna.

The direction detection unit 12 detects a direction for the millimeter-wave radar 10 to irradiate the subject 150 with millimeter waves. For this purpose, in the direction detection unit 12, detection by millimeter waves output from a multi-channeled millimeter-wave radar and detection by Light Detection and Ranging (LiDAR) may be performed, and detection support by imaging by a camera or the like may be performed.

The direction detection unit 12, as an example, detects, as a direction of the neck, a direction of a vicinity of a point, which is most recessed in a viewpoint as viewed from the millimeter-wave radar 10, on a surface on an occipital side from the head to a torso of the subject 150. When the neck of the subject 150 is viewed from behind the neck of the subject 150, the neck is recessed toward a front side of the subject 150 along an arch shape of a cervical spine. Therefore, the direction detection unit 12 may detect, as a direction in which millimeter waves are to be radiated, a direction in a vicinity of a point that is most convex in a direction from the millimeter-wave radar 10, which is a laser light source that views the neck from behind the neck, to the neck, that is, a point, which is most recessed in the viewpoint as viewed from the millimeter-wave radar 10, on the neck.

Alternatively, the direction detection unit 12 detects, as the direction of the neck, a direction from the millimeter-wave radar 10 toward a center point in a width of a portion, in which the width is narrowest, on the neck of the subject 150, the width being defined in a direction intersecting a direction from the head of the subject 150 to the torso of the subject 150. Here, the direction from the head of the subject 150 toward the torso of the subject 150 may be an extending direction of the cervical spine. Further, the direction intersecting the direction from the head of the subject 150 toward the torso of the subject 150 may be a direction perpendicular to the extending direction of the cervical spine.

Alternatively, the direction detection unit 12 may detect, as the direction of the neck, a direction of a vicinity of a point, which is most recessed in a viewpoint as viewed from the millimeter-wave radar 10 toward the body surface, on the body surface on the occipital side from the head to the torso of the subject 150. Further, the direction detection unit 12 may regard, as the head, a vicinity of a point above the most recessed point in a gravity direction, and may detect, as a back portion, a vicinity of a point below the most recessed point in the gravity direction. Therefore, the direction detection unit 12 may detect, as the direction of the neck, the direction that is most convex toward the direction from the millimeter-wave radar 10, which is the laser light source that views the neck of the subject 150 from behind the subject 150, to the neck, that is, the direction in the vicinity of the point, which is most recessed in the viewpoint as viewed from the millimeter-wave radar 10, on the neck of the subject 150. Further, the direction detection unit 12 may calculate, from the direction of the neck, an upper head position or a lower back position corresponding to a general human body size, by using distance information from the millimeter-wave radar 10 to the neck. Further, the direction detection unit 12 may detect the calculated direction as the direction in which millimeter waves are to be radiated.

When the millimeter-wave radar 10 irradiates the neck of the subject 150 from the back of the subject 150, the width in the direction intersecting the direction from the head of the subject 150 toward the torso of the subject 150 corresponds to a width in a viewpoint as the neck of the subject 150 is viewed from behind the subject 150.

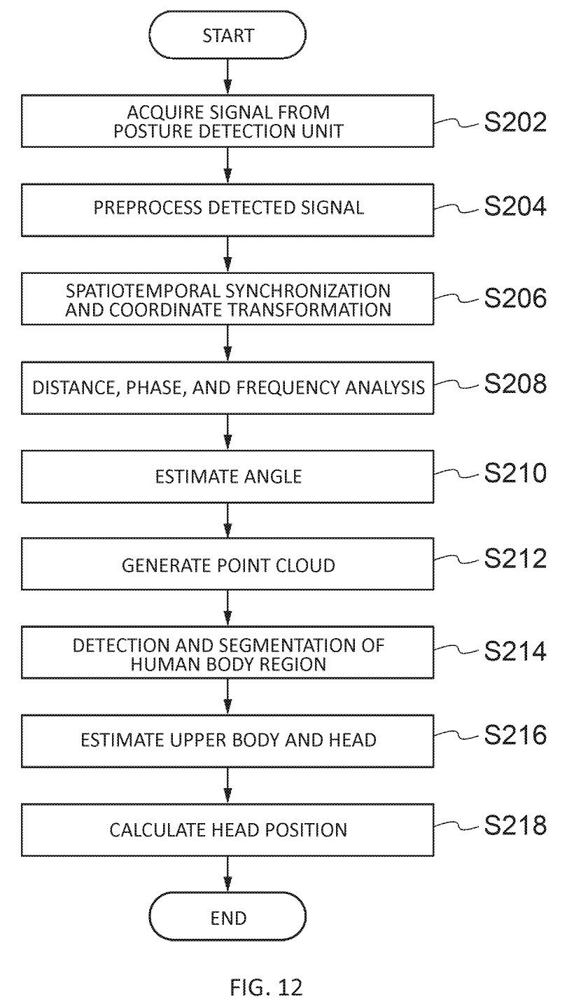

The posture detection unit 14 detects a posture of the subject 150. A detailed operation of the posture detection unit 14 will be described later with reference to a flowchart of FIG. 12.

The chewing count detection unit 16 detects a chewing count during a meal of the subject 150. Here, the chewing count detection unit 16 is set as a detection unit for the chewing count as detecting at least a count of chews; however, the chewing count detection unit 16 may detect other information such as a chewing tempo of chewing of the subject 150 and a chewing time between swallows.

As an example, the chewing count detection unit 16 may be implemented by a sensor that detects a motion of a reference point of a temporomandibular joint or a mandible by using millimeter waves. However, the chewing count detection unit 16 is not limited to the sensor using millimeter waves, and as another example, the chewing count detection unit 16 may include a camera, capture a moving image of a face of the subject 150, and perform chewing count detection by image analysis for the motion of the mandible. As still another example, the chewing count detection unit 16 may be a sensor that is worn on an ear and detects the motion of the temporomandibular joint or the like around an auricle.

The control unit 20 includes a signal acquisition unit 22, a signal processing unit 24, an evaluation unit 26, a radar control unit 28, a posture acquisition unit 30, a correction unit 32, and a biological information acquisition unit 34. In particular, a more detailed configuration of the signal acquisition unit 22 and the signal processing unit 24 will be described later with reference to FIG. 3.

The signal acquisition unit 22 acquires a signal that is obtained by receiving reflected waves of millimeter waves radiated by the millimeter-wave radar 10 to the neck of the subject 150, and indicates a temporal change in a distance from the millimeter-wave radar 10 to the neck. The signal is acquired from the millimeter-wave radar 10. The signal acquisition unit 22 outputs the acquired signal to the signal processing unit 24.

The signal processing unit 24 processes the signal acquired by the signal acquisition unit 22 to perform preprocessing for the evaluation unit 26 to evaluate the aspiration state and the swallowing function of the subject 150. The signal acquisition unit 22 performs component extraction, noise removal, and signal enhancement on the acquired signal, and performs processing so that the evaluation unit 26 can easily evaluate presence or absence of the aspiration state of the subject 150, and the swallowing ability.

The evaluation unit 26 evaluates at least one of the swallowing function or the aspiration state of the subject, based on the temporal change in the distance indicated by the signal. The evaluation unit 26 may evaluate at least one of the swallowing function or the aspiration state of the subject 150 by using machine learning. The evaluation unit 26 may numerically evaluate the swallowing function of the subject 150 in a stepwise manner according to various risk factors and the like, for example, by assigning a high number when the swallowing function is at a high level and assigning a low numerical value when the swallowing function is at a low level. The evaluation unit 26 may evaluate the swallowing function by statistically analyzing a waveform indicating the temporal change in the distance included within a predetermined period to identify a peak in the waveform, and using a “swallowing function index” that is a numerical value to which a higher number is assigned when the swallowing function is at a high level and to which a lower numerical value is assigned when the swallowing function is at a low level.

The evaluation unit 26 may evaluate at least one of the swallowing function or the aspiration state of the subject 150 by using a machine learning model that has been machine-trained with the temporal change in the distance from the millimeter-wave radar 10 to the neck of the subject 150 as an input and at least one of the swallowing function or the aspiration state as an output to estimate at least one of the swallowing function or the aspiration state of the subject 150 with respect to the temporal change in the distance indicated by the signal detected by the signal acquisition unit 22. A type of the machine learning model is not limited as long as the machine learning model is a model suitable for evaluating at least one of the swallowing function or the aspiration state of the subject 150. As the machine learning model, any suitable algorithm such as a support vector machine (SVM), multiple regression analysis, a random forest, or a neural network, in particular deep learning using a deep neural network, may be used.

The radar control unit 28 outputs, to the millimeter-wave radar 10, a control signal for controlling a direction in which the millimeter-wave radar 10 radiates millimeter waves, based on the direction of the neck of the subject 150 detected by the direction detection unit 12. The control signal of the radar control unit 28 may be used as an input for transmit beamforming (for example, phase addition processing) as directivity control on a transmission side of the millimeter-wave radar 10 and/or for mechanical directivity control for directing the millimeter-wave radar 10.

The posture acquisition unit 30 acquires a signal regarding the posture of the subject 150 detected by the posture detection unit 14, and reads posture information of the subject 150 from the detected signal. Processing in the posture acquisition unit 30 will be described later with reference to FIG. 12. For example, when the back of the subject 150 is rounded or their feet are not on the ground during a meal, the aspiration risk increases. The evaluation unit 26 may evaluate at least one of the swallowing function or the aspiration state of the subject, based on the posture information acquired by the posture acquisition unit 30, further based on the posture information.

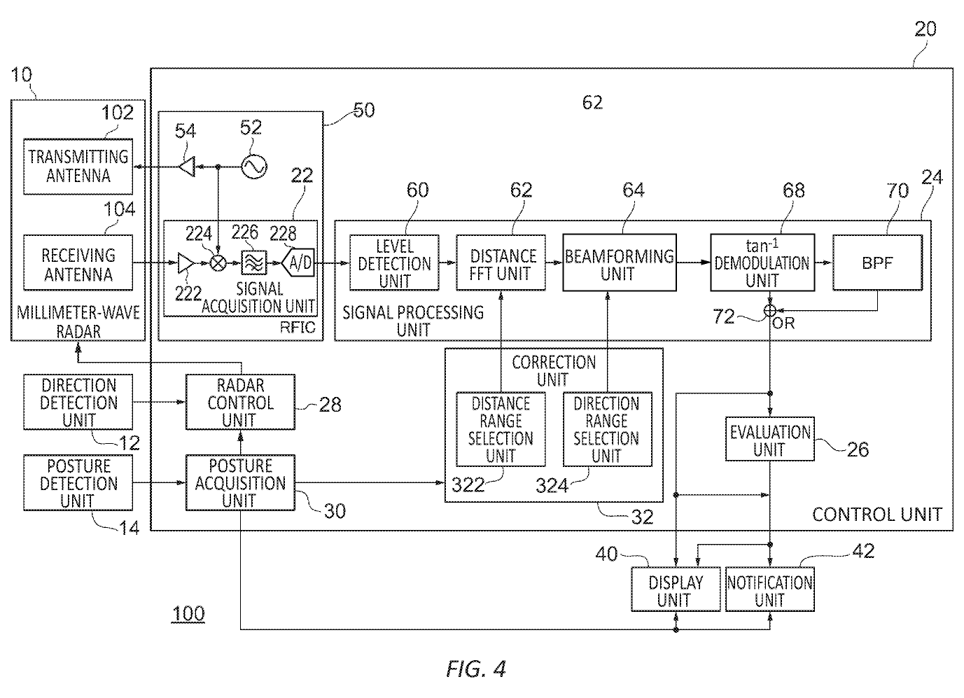

The radar control unit 28 may correct control of the transmit beamforming for the direction of the neck of the subject 150 detected by the direction detection unit 12, further based on the posture information acquired by the posture acquisition unit 30. The millimeter-wave radar 10 includes a transmitting antenna 102 to be described later with reference to FIG. 4. The transmitting antenna 102 includes a phased antenna array in which a plurality of antenna elements are disposed in an array at predetermined intervals.

The control signal output by the radar control unit 28 may be a signal for electronically controlling a main direction of a beam through interference by adjusting a transmission signal in the transmitting antenna 102 according to a desired irradiation direction. For example, when the subject 150 tilts the upper body in a left-right direction by an angle θ and the neck is tilted by the angle θ, the radar control unit 28 performs control through beamforming to direct the main beam direction in a direction tilted by the angle θ according to a tilt angle of the neck.

In this case, an interval between antenna elements in the phased antenna array is d, a wavelength of millimeter waves to be used is λ, and an angle of a beam irradiation direction is θ. A propagation distance difference in an θ direction of signal waves transmitted from each antenna element is d×sinθ. In this case, the radar control unit 28 adds a phase shift of n×Δφ to an n-th element of the antenna array by giving a phase difference Δφ = (2πd×sinθ)/λ between adjacent antenna elements. Therefore, the radar control unit 28 generates a control signal indicating the phase difference Δφ to be given to each antenna element, and transmits the control signal to each antenna element. Thus, beams in the angle θ direction are aligned in equal phase, and interference waves in the angle θ direction reach maximum amplitude.

Conversely, in directions other than the angle θ direction, arrival phases of waves are not aligned and enhancement due to interference does not occur, and thus beams are concentrated in the angle θ direction. The radar control unit 28 can perform high-speed and dynamic target tracking by using an interference effect of waves to electronically control the beam direction, and can control the millimeter-wave radar 10 to intensively radiate millimeter waves in the direction of the neck.

Further, in addition to the transmit beamforming control, the radar control unit 28 may output, to a mechanical adjustment mechanism for the irradiation direction such as a pan/tilt mechanism, a gimbal mechanism, a rotation motor, or an arm mechanism, a control signal for controlling driving of these. The radar control unit 28 calculates a target direction based on the direction detected by the direction detection unit 12 and the angle indicated by the posture information, and outputs, to the adjustment mechanism, a drive amount (a rotation angle of a motor, etc.) corresponding thereto as a control signal. The control signal may be indicated by a PWM signal or a position command value, or the like. By such directivity control by mechanically rotating or tilting a main body of the millimeter-wave radar 10, it is possible to set an initial irradiation direction or to complement coverage of the directivity control by electronic beamforming.

The biological information acquisition unit 34 acquires biological information regarding a living body of the subject, the biological information including predetermined items that affect the swallowing function of the subject. The biological information acquired by the biological information acquisition unit 34 may include personal information serving as health indices, such as age, gender, weight, height, body fat percentage, or muscle mass. The evaluation unit 26 may evaluate at least one of the swallowing function or the aspiration state of the subject 150 further based on the biological information acquired by the biological information acquisition unit 34.

For example, when age advances, the swallowing ability tends to decline due to muscle weakness in peripharyngeal muscles, etc. For example, the evaluation unit 26 may divide the age of the subject 150 into, for example, less than 60 years old, 60 to 65 years old, 65 years old to 70 years old, and 70 years old to 75 years old, and may set a stepwise numerical evaluation of the swallowing function to be lower as an age range becomes higher (e.g., lowering it by 0.5 rank as the subject falls into a range that is 5 years older). In addition, regarding the gender, it is known that, in males, a decrease in the position of the pharynx and a decrease in maximum tongue pressure occur from an earlier period than in females, and the decline in swallowing function is more likely to occur than in females. Therefore, if the subject 150 is a male, the evaluation unit 26 may perform evaluation such that the stepwise numerical evaluation of the swallowing function is set to a numerical value lower than that for a female (for example, lowering it by one rank, 0.5 rank, or the like). In addition, as the body fat percentage is higher, a risk of sleep apnea syndrome and the like increases, and it has been pointed out that respiratory disorders are also related to swallowing disorders. Therefore, the evaluation unit 26 may evaluate whether or not the subject 150 is in an obese state based on the weight, the height, and the body fat percentage, and when the subject 150 is determined to be in an obese state, the evaluation unit 26 may perform evaluation with obesity as a risk factor in the evaluation of the swallowing function, such that the stepwise numerical evaluation of the swallowing function is set to a low numerical value (for example, lowering it by one rank, two ranks, or the like) as compared with a case where the subject 150 is not in an obese state.

Furthermore, in recent years, a relationship between sarcopenia, which refers to a state of an age-related decline in muscle mass, and swallowing disorders has also been pointed out. In particular, a functional decline of masticatory muscles and peripharyngeal muscles can directly cause a decline in swallowing function. Therefore, the evaluation unit 26 may evaluate the decline in muscle mass as a risk factor for the decline in swallowing function. For example, if muscle mass measured by a body composition meter is less than a threshold, the evaluation unit 26 may perform evaluation such that the stepwise numerical evaluation of the swallowing function is set to a low numerical value (for example, lowering it by one rank, two ranks, or the like).

Furthermore, when sarcopenia occurs, a BMI value calculated from height and weight decreases. Therefore, the height and weight may be used not only for evaluation of obesity but also for evaluation of whether or not sarcopenia has occurred, and the evaluation unit 26 may evaluate the swallowing function of the subject 150 based on whether or not the height and weight of the subject 150 are within appropriate ranges, whether or not a rapid change in weight has occurred, and the like.

The evaluation unit 26 may provide a threshold for BMI, and when the BMI of the subject 150 is less than the threshold, sarcopenia may have occurred, and, using this as a risk factor, the evaluation unit 26 may perform evaluation such that the stepwise numerical evaluation of the swallowing function is set to a low numerical value (for example, lowering it by one rank, two ranks, or the like) as compared with a case where the BMI of the subject 150 is equal to or greater than the threshold. If there is weight loss of 5 kg or more in half a year, the evaluation unit 26 may perform evaluation such that the stepwise numerical evaluation of the swallowing function is set to a low numerical value (for example, lowering it by one rank or the like). In addition, if the BMI of the subject 150 is equal to or greater than the threshold indicating obesity, the evaluation unit 26 may perform evaluation such that the stepwise numerical evaluation of the swallowing function is set to a low numerical value (for example, lowering it by one rank, two ranks, or the like). The evaluation unit 26 may evaluate the swallowing function of the subject 150 using a score in the numerical evaluation of the swallowing function, based on a stored table indicating a correspondence relationship between each item of biological information and a variation value of a numerical evaluation of each item. The evaluation unit 26 may evaluate the swallowing function of the subject 150 based on a correspondence relationship between each item of biological information and pre-stored variations in the numerical evaluation of the swallowing function.

The biological information acquired by the biological information acquisition unit 34 may include medical records including predetermined items that affect the swallowing function of the subject. Here, the medical records are information of the subject 150 acquired by examinations and the like in a medical institution, and may be, for example, information acquired by periodic examinations such as a video endoscopic examination of swallowing (VE) or a video fluoroscopic examination of swallowing (VF). Furthermore, the medical records may include information regarding a cognitive function of the subject 150, or medical history, etc. Regarding such information, for example, with the cognitive function, information periodically acquired by a simple test by a nursing care facility or the like may be associated, and with memory history, information based on self-report by subject 150 or report by a family of the subject 150 may be associated, but the information may also be acquired from a system of a medical institution.

For example, as the medical history affecting a swallowing function, masses, tumors, or trauma in the oral cavity, pharynx or esophagus, chronic obstructive pulmonary disease (COPD), tracheal intubation, and stroke, etc. are known. The medical histories of these diseases are risk factors for the decline in swallowing function. For the subject 150 having a medical history of a predetermined disease, the evaluation unit 26 may, for example, assign a score for each disease and perform evaluation such that the numerical evaluation of the swallowing function is set to a low numerical value, thereby shifting the evaluation such that the risk of the decline in swallowing function is high.

Furthermore, medical records of dentistry or oral surgery, etc., such as an occlusal condition and whether or not oral care are sufficient, have high relevance to the swallowing function. In addition, a decline in cognitive function tends to cause swallowing disorders. Evaluation of the cognitive function of the subject 150 may be performed by a simple test or the like at a nursing care facility or the like, but may be performed at a specialized medical institution, and may be stored as a medical record together with results of Magnetic Resonance Imaging (MRI), etc. The biological information may include medical records of medical institutions corresponding to respective pieces of biological information, and the evaluation unit 26 may assign a score according to the risk factor for the swallowing function of the subject 150, based on the medical records, and may perform evaluation such that the numerical evaluation of the swallowing function is set to be low when the risk factor exists.

For example, the evaluation system 100 may store, as medical history of the medical records, a table listing a score reduction value for each disease such that a disease having a greater influence on the decline in swallowing function (an increase in aspiration risk) has a larger score reduction. The evaluation unit 26 may evaluate the swallowing function of the subject 150 using the score in the numerical evaluation of the swallowing function, based on the medical history of medical records and a stored table indicating a correspondence relationship between the medical history of medical records and a variation value of numerical evaluation for each item of the medical history of medical records. In this manner, the evaluation unit 26 may evaluate the swallowing function of the subject 150 based on a correspondence relationship between the medical history of the medical records and the pre-stored variations in the numerical evaluation of the swallowing function.

A schedule acquisition unit 36 acquires a schedule indicating at least one of a mealtime provided to a subject or a sleep time of the subject. For example, during a meal or shortly after the meal, aspiration due to reflux of a bolus from the esophagus or stomach is likely to occur. Therefore, for example, when the evaluation system 100 is installed on a chair of a dining room, in a case where the evaluation unit 26 evaluates whether or not the subject 150 is in an aspiration state, even if another evaluation parameter indicates a boundary value of whether or not the subject 150 is in an aspiration state, the evaluation unit 26 may determine that the subject 150 is in an aspiration state if it is during mealtime. In this manner, the evaluation unit 26 may evaluate at least one of the swallowing function or the aspiration state of the subject 150 further based on the schedule of mealtime.

In addition, by grasping a sleep schedule of the subject 150, when the subject 150 is lying in a same posture on the bed 200 (a posture in a recumbent position) in a time period scheduled for sleep and in a time period not scheduled for sleep, it can be estimated that the time period scheduled for sleep has a higher possibility of being a sleep state (consciousness is not awake). In the sleep state, a human experiences a decrease in swallowing reflex, which is a reflexive action of peripharyngeal muscles that occurs spontaneously when swallowing food or drink, and cough reflex, which is a response that causes coughing by contraction of respiratory muscles, etc. in response to stimulation in the airway and eliminates a foreign substance. In addition, while the subject 150 is sleeping, cough, sputum, or the like may flow into the airway, resulting in an aspiration state in some cases. Therefore, even when the subject 150 is lying down in a same manner, if it is possible to grasp whether or not the subject 150 is in a sleep state, the evaluation unit 26 can evaluate the aspiration risk more accurately.

In addition, it is considered that a posture in which a human is lying down (a supine position, a lateral position, and a prone position, etc.) affects the aspiration risk. For example, it is known that, in the supine position, a tongue base tends to fall backward due to gravity, compressing the airway, which makes respiration shallow and the cough reflex readily suppressed, and reflux in the esophagus also occurs more easily than in the lateral position. Therefore, when an evaluation result of a boundary between an aspiration state and a non-aspiration state is obtained from evaluation of vibration corresponding to, for example, the swallowing sound of the neck, etc., if the subject 150 is in the time period scheduled for sleep and is in the supine position, since the aspiration risk becomes higher, the evaluation unit 26 may shift the evaluation such that the subject 150 is in an aspiration state. On the other hand, when the subject 150 is in the lateral position and is outside the scheduled time of the sleep schedule, the evaluation unit 26 may continue measurement further to evaluate the subject 150 as being in an aspiration state after accumulating more information determined to be aspiration. Therefore, based on the sleep schedule of the subject 150, the evaluation unit 26 may perform evaluation on at least one of the swallowing function or the aspiration state of the subject 150 such that, at the time scheduled for the sleep of the subject 150, the swallowing function has further declined (for example, lowering the numerical evaluation by one rank or two ranks) and a risk of being in an aspiration state increases.

A chewing count acquisition unit 38 acquires a chewing count during a meal of the subject 150. The chewing count is one element of a chewing function of the subject 150, and is also closely related to the swallowing ability. For example, if the chewing count is large and the chewing function is at a high level, food is finely crushed and is well mixed with saliva, thereby forming a soft bolus.

Eating and swallowing can be divided into five phases: an anticipatory phase until food is recognized and taken into a mouth, a preparatory phase in which the food is chewed and mixed with saliva to form a bolus, an oral phase in which the bolus is sent into a pharynx by movement of the tongue or the like, a pharyngeal phase in which the bolus is carried from the pharynx to an esophagus, and an esophageal phase in which the bolus is carried to a stomach. Swallowing can be considered as corresponding to two phases among these, namely, the pharyngeal phase and the esophageal phase. Chewing is performed in the preparatory phase for preparation for the swallowing. The softer the bolus is, the easier it is to swallow the food, and if normal coordination of perioral muscles in chewing is not performed, transfer of the bolus is also not performed well, and swallowing disorders are likely to be caused. Therefore, a decline in chewing function is also closely related to the decline in swallowing function.

The evaluation unit 26 may evaluate at least one of the swallowing function or the aspiration state of the subject, further based on information on the swallowing function of the subject 150, such as the chewing count. For example, the evaluation unit 26 may numerically evaluate the chewing function, and when the numerical evaluation of the chewing function is lower than a predetermined threshold, use this as a risk factor to perform evaluation such that the numerical evaluation of the swallowing function is set to a low numerical value (for example, lowering it by one rank, two ranks, or the like).

An example of the machine learning model in evaluation of the swallowing function performed by the evaluation unit 26 when these evaluation parameters are present will be described. For example, as an input of machine learning in a training phase, features are extracted from explanatory variables by using, as an explanatory variable vector, age, gender, weight, height, body fat percentage, muscle mass, periodic examination results, medical history, medical records such as dentistry or oral surgery, cognitive function (and numerical evaluation scores thereof), and the like of the subject 150. Further, the evaluation unit 26 may train a model by using teacher labels in which "1" is assigned to a case where an aspiration state is not detected in the video endoscopic examination of swallowing within three years of the subject 150, and "0" is assigned to a case where an aspiration state is detected in the video endoscopic examination of swallowing within three years.

In an inference phase, the evaluation unit 26 acquires swallowing-related data on the subject 150 similar to that listed as the explanatory variable vector in the training phase. The machine learning model may calculate "a probability of not causing aspiration in the video endoscopic examination of swallowing within three years" by inputting these input variables to the machine learning model and mapping them to a continuous value of 0 or more and 1 or less.

The display unit 40 displays an evaluation result by the evaluation unit 26. The display unit 40 may be a monitor that visually displays the evaluation result.

The notification unit 42 provides notification of the evaluation result by the evaluation unit 26. The notification unit 42 may function as an alert apparatus that issues a warning when a part of numerical values in the evaluation result by the evaluation unit 26 exceeds a preset threshold. The alert may be a visual alert transmitted to the display unit 40, and may be emitted as an acoustic signal serving as the alert.

The notification unit 42 may include a communication function of transmitting and recording the evaluation result and measurement data to and in an electronic medical record or a medical record system. The medical record system may be, for example, the server 162 connected to the evaluation system 100 via the network 160 described with reference to FIG. 1.

FIG. 4 is a diagram for describing an example of a detailed configuration of some blocks in the evaluation system 100 shown in FIG. 3. The evaluation system 100 includes the millimeter-wave radar 10, the direction detection unit 12, the posture detection unit 14, the control unit 20, the display unit 40, and the notification unit 42. In FIG. 4, with respect to the configuration described with reference to FIG. 3, configurations of the millimeter-wave radar 10, the signal acquisition unit 22, the signal processing unit 24, and the correction unit 32 are depicted in more detail.

The millimeter-wave radar 10 includes the transmitting antenna 102 and a receiving antenna 104. An irradiation direction of millimeter waves by the millimeter-wave radar 10 may be adjusted by the radar control unit 28 controlling the millimeter-wave radar 10 based on the direction detected by the direction detection unit 12.

The RFIC 50 includes a portion that generates millimeter waves to be transmitted from the transmitting antenna 102, and the signal acquisition unit 22 that acquires, from millimeter waves input from the receiving antenna 104, a variation in the distance to the neck of the subject 150. The millimeter waves output from the transmitting antenna 102 are generated via an AC power supply 52 and an amplifier 54.

The AC power supply 52 is a power supply for transmitting millimeter waves. The AC power supply 52 is represented by a circuit symbol of a power supply for simplicity, but, for example, when the millimeter-wave radar 10 is an FMCW-type millimeter-wave radar, the AC power supply 52 may be configured with a power supply that generates a chirp signal and a Voltage Controlled Oscillator (VCO) that is an oscillator having a function of changing a frequency.

The amplifier 54 is a power amplifier that amplifies an AC voltage output from a power supply such that the transmitting antenna 102 can output millimeter waves having sufficient intensity.

The signal acquisition unit 22 acquires a signal input from the receiving antenna 104, performs Analog to Digital (A/D) conversion, and outputs the signal. The signal acquisition unit 22 includes an amplifier 222, a multiplier 224, a low-pass filter 226, and an ADC 228 (Analog to Digital Converter).

The amplifier 222 amplifies the signal input from the receiving antenna 104. As an example, the amplifier 222 is a low noise amplifier (LNA).

The multiplier 224 multiplies and combines output waves before being amplified by the amplifier 54, to generate an intermediate frequency (IF) signal in order to perform, in analysis of reflected waves, analysis based on a difference from irradiation waves.

The low-pass filter 226 is a filter that functions as an anti-aliasing filter for a signal input to the ADC 228.

The ADC 228 performs Analog to Digital conversion (AD Conversion) on the acquired analog signal and outputs a digital signal. Digitizing the acquired signal facilitates signal processing by the signal processing unit 24 and evaluation by the evaluation unit 26.

Next, processing performed by the signal processing unit 24 will be described. The signal processing unit 24 performs preprocessing on the digital signal acquired by the signal acquisition unit 22 to enable the evaluation unit 26 to perform evaluation. The signal processing unit 24 includes a level detection unit 60, a distance FFT unit 62, a beamforming unit 64, a tan⁻¹ demodulation unit 68, a bandpass filter 70, and a multiplier 72.

The level detection unit 60 performs signal extraction by performing filtering on the acquired signal according to the signal level. The level detection unit 60 performs noise removal and exclusion of an unnecessary region by limiting range information of the amplitude to a specific range.

The distance FFT unit 62 can perform processing of performing a distance Range Fast Fourier Transformation (FFT) to acquire a distance power spectrum in a frequency domain and estimating a distance to a target from a frequency difference between a transmitted signal and a received signal.

For example, when an FMCW-type chirp signal is used as millimeter waves, the millimeter waves change in frequency during a chirp time. A propagation delay of a reflected signal with respect to a transmission signal, appearing in an intermediate frequency signal, appears as a frequency difference. Using f_{IF} as a frequency component included in the received IF signal, S as a slope (frequency change rate) of the chirp signal, and c as speed of light, a distance R to a reflection target satisfies R = (c×f_{IF})/2S. By deriving a signal intensity of each frequency component for the IF signal and deriving a reflection intensity distributed in a distance direction, the distance to the reflection target can be calculated. Thus, by analyzing a frequency spectrum of the reflected signal by Fourier transform of the IF signal, distance information of the target can be obtained. This enables the position of the neck, which is a measurement target, to be evaluated based on the signal acquired from the signal acquisition unit 22.

In addition, vibration of the neck associated with swallowing appears as a variation in the distance to the neck. With an output of the distance FFT unit 62, the evaluation system 100 can evaluate vibration components of the neck associated with swallowing for each frequency. The vibration of the neck associated with swallowing provides an output similar to a swallowing sound. Although the evaluation system 100 uses the neck as an example of the body surface for which vibration associated with swallowing is detected, the body surface may be any portion where the swallowing sound propagates through the body and appears on the body surface, and may be, for example, the body surface such as the head, the back, or the chest.

Here, in the present embodiment, the term "FFT" is used; however, the distance FFT unit 62 only needs to perform spectrum analysis by performing time and frequency conversion, and conversion performed in the distance FFT unit 62 is not limited to Fourier transform, and wavelet transform may be performed. By performing the wavelet transform, a scene in which a distance changes suddenly with time, that is, a temporally localized motion (a motion appearing only momentarily) and the like can be extracted with higher accuracy than windowed Fourier transform in FFT.

The beamforming unit 64 extracts signal components for each angle. Specifically, this corresponds to receive beamforming processing as directivity control on a receiving side that analyzes a spatial direction component (the angle of the target) by using phase differences of a plurality of channels of signals obtained by the antenna array. Note that, instead of processing of extracting, by performing a weighted sum, signal components for each angle by beamforming, angle FFT may be performed to perform processing of spatially performing FFT processing on data of a plurality of channels to identify the angle. As already described, when an interval between antenna elements in a phased array antenna is d, if the wavelength of millimeter waves is λ and an arrival direction of reflected waves is an angle θ, a phase difference Δφ of received signals of respective receiving antenna elements is given by Δφ = (2πd×sinθ)/λ. The beamforming unit 64 can identify the arrival direction of the reflected waves by comparing combined-signal intensities of the reflected waves received by respective receiving antenna elements. Beamforming processing as directivity control may be performed not only on the receiving side but also in a Multi-Input Multi-Output radar (MIMO radar) using both transmission and reception.

The posture detection unit 14 detects the posture of the subject 150, and the posture acquisition unit 30 outputs posture information based on a signal detected by the posture detection unit 14. The posture information by the posture acquisition unit 30 may be transmitted to the display unit 40 and the notification unit 42 together with the evaluation result by the evaluation unit 26. When the posture of the subject 150 during a meal is inappropriate and is a posture that is likely to cause aspiration, the display unit 40 and the notification unit 42 may output a warning and an instruction for posture adjustment. Detailed processing regarding posture detection by the posture detection unit 14 and acquisition of the posture information by the posture acquisition unit 30 will be described later with reference to the flowchart of FIG. 12.

The correction unit 32 corrects a distance component and an angle component of a signal, based on the posture information acquired by the posture acquisition unit 30. Specifically, for example, in an initial state of measurement, when the millimeter-wave radar 10 is located behind the head and the neck of the subject 150, the correction unit 32 applies correction to a distribution in the distance direction obtained by distance FFT in accordance with a tilt in a front-rear direction of the head and the neck of the subject 150, or a posture change of forward leaning and backward leaning of a state of the subject 150. On the other hand, the correction unit 32 performs correction on an angle-direction component obtained by beamforming in accordance with displacement of the posture of the subject 150 in the left-right direction or an up-down direction.

The correction unit 32 improves accuracy of signal extraction around the neck of the subject 150 by performing correction on the processing performed by the distance FFT unit 62 and the beamforming unit 64 to dynamically correct a spatial component of the signal. In particular, even when the subject 150 changes the posture and deviates from the front of the millimeter-wave radar 10, or even when the subject 150 makes a turn over or the like, a signal processing target can be effectively enhanced. The correction unit 32 includes a distance range selection unit 322 and a direction range selection unit 324.

The distance range selection unit 322 adjusts weighting of the signal output by the distance FFT unit 62, based on the posture information acquired by the posture acquisition unit 30. For example, in processing in the distance FFT, when FMCW millimeter waves are used, the chirp signal has only a finite bandwidth, and a window function in the FFT performs processing in a finite-size window, etc., so that, even when there is originally only one reflection point, a spectral distribution of the signal appears in a form distributed over a plurality of distance ranges. Here, “weighting” means processing in which, for each signal processed in the distance FFT separately for each distance range from the millimeter-wave radar 10, a coefficient is multiplied, thereby increasing an amplitude of a signal to be enhanced and decreasing a signal at a distance that is not to be enhanced.

For example, description will be made of a case where, in the initial state of measurement as in the example described above, the millimeter-wave radar 10 is located behind the head and the neck of the subject 150. In this case, when the subject 150 takes a forward-leaning posture, the neck moves forward, and thus the distance from the millimeter-wave radar 10 to the neck becomes relatively longer. Therefore, the distance range selection unit 322 corrects a weight distribution in the distance direction such that a relatively high weight is applied to a distance component on a far-distance side. On the other hand, when the subject 150 leans backward, for example, by attempting to lean against the chair, a position of the neck approaches the millimeter-wave radar 10. In this case, the weight distribution in the distance direction is corrected such that a relatively high weight is applied to a component on a short-distance side. By such processing, it is possible to make a signal component in a distance band in which the neck is highly likely to exist stand out, suppress other signals, and suppress a signal component which is based on noise or a non-target component, etc.

As described above, the distance range selection unit 322 may calculate an estimated position of the neck of the subject 150 as viewed from the millimeter-wave radar 10, based on posture information by the posture acquisition unit 30, and may emphasize a weight around a distance center by using a weight distribution that has the distance center as a peak and attenuates in accordance with a distance difference from the distance center. Thus, weighting of signal components corresponding to a distance range at a neck position with respect to the millimeter-wave radar 10, which is estimated based on the posture information, is corrected.

The direction range selection unit 324 adjusts weighting of the signal output by the beamforming unit 64, based on the posture information acquired by the posture acquisition unit 30. For example, a case where, in the initial state of measurement as in the example described above, the millimeter-wave radar 10 is located behind the head and the neck of the subject 150 will be described. In this case, the direction range selection unit 324 may adjust a beamforming weight coefficient such that, in receive beamforming, a higher weight is applied to an angle component in a displacement direction in accordance with the displacement of the posture of the subject 150 in the left-right direction or the up-down direction, thereby enhancing the angle-direction component in a desired range. Thus, the weighting of the signal components corresponding to the angle range at the neck position with respect to the millimeter-wave radar 10, which is estimated based on the posture information is corrected.

The tan⁻¹ demodulation unit 68 continuously calculates tan⁻¹ (inverse tangent, arctangent) of an IQ signal. The signal can be obtained in a form of A×exp(j×φ(t)) = I(t) + j*Q(t) as a complex-number mode. Here, t is time, A is an amplitude, φ is a phase, j is an imaginary number, I is a real component of the signal, and Q is an imaginary component of the signal. In this case, the tan⁻¹ demodulation unit 68 extracts a phase change by φ = tan⁻¹(Q(t)/I(t)). Thus, the signal processing unit 24 can extract, with high resolution, the smaller displacement that appears in a phase component and has a non-stationary velocity change.

The bandpass filter 70 is a filter for removing noise in a specific frequency band and enhancing a signal. The multiplier 72 multiplies the signal output by the tan⁻¹ demodulation unit 68 and the signal output by the bandpass filter 70, and outputs a signal from which noise has been removed.

The evaluation unit 26 evaluates, based on the signal processed by the signal processing unit 24, whether or not the subject 150 is in an aspiration state, and the swallowing function of the subject 150. The evaluation unit 26 outputs an evaluation result to the display unit 40 and the notification unit 42. Further, the evaluation system 100 may output the evaluation result by the evaluation unit 26 to the external server 162, etc. via the network 160 described with reference to FIG. 1.

Furthermore, the evaluation result by the evaluation unit 26 may be stored in a storage unit (not illustrated) inside or outside the evaluation system 100. Accordingly, the evaluation system 100 may statistically analyze accumulated evaluation results to continuously optimize an aspiration risk evaluation algorithm by the evaluation unit 26.

The display unit 40 and the notification unit 42 may output, visually and by voice, the evaluation result by the evaluation unit 26 and, as necessary, a waveform itself of data processed by the signal processing unit 24, and the like. The notification unit 42 may provide real-time voice support and instructions to the subject 150 in accordance with the evaluation results of the aspiration state and the swallowing function of the subject 150.

In the evaluation system 100 according to the present embodiment, the swallowing function and the aspiration state of the subject 150 can be evaluated by the non-contact and low-noise method, based on the vibrations and displacements and the like of the neck accompanying the swallowing of the subject 150, by using millimeter waves. Accordingly, the swallowing function and the aspiration state of the subject 150 can be evaluated with a small burden on the subject 150 and with high accuracy. The “evaluation apparatus” may be implemented by the control unit 20 according to the present embodiment.

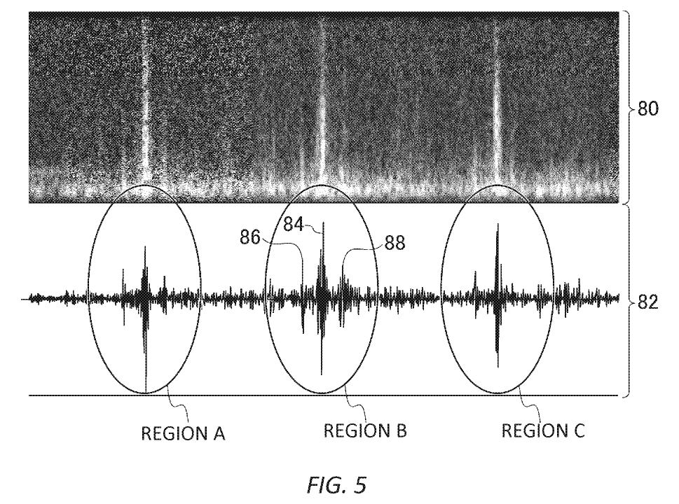

FIG. 5 illustrates an example of a waveform 80 acquired by millimeter waves and a waveform 82 of a signal obtained by processing the waveform 80.

The waveform 82 is an example of a waveform obtained by performing noise removal and smoothing via the bandpass filter 70 and the multiplier 72 after the tan⁻¹ demodulation unit 68, and further performing processing of enhancing a signal change by performing differential amplification and the like. A pattern of the waveform 82 is similar to a waveform pattern that has been obtained by detecting sounds when the subject 150 swallows and respires. On the other hand, the waveform 80 indicates a time-dependent change of a spectrum obtained by performing FFT on the waveform 82 for each section. By performing detection using millimeter waves, the evaluation system 100 becomes capable of detecting a signal accompanying swallowing or respiration even when the millimeter-wave radar 10, which is the detection portion, is separated from the subject 150 by a longer distance than in a case of using sound, and becomes capable of acquiring the waveform 82 as detection having high noise resistance. In this manner, in the evaluation system 100, signal peaks corresponding to a swallowing sound and a respiration sound can be acquired.

The evaluation unit 26 may identify, in a waveform indicating a temporal change in distance, a plurality of peaks indicating a change in distance exceeding a predetermined threshold included within a predetermined period. This threshold may be a threshold exceeding an average of lowest ones of the plurality of peaks, and is an example of a “first threshold”.

Peaks accompanying swallowing have three peaks 84, 86, and 88. Among these, the peak 84 has a largest amplitude, and the peak 86 and the peak 88 have amplitudes smaller than the amplitude of the peak 84.

The evaluation unit 26 may detect a plurality of peaks 84 by using a predetermined threshold as a threshold larger than the first threshold among the three peaks. In addition, the evaluation unit 26 may identify the peaks 84, 86, and 88 as the peaks accompanying swallowing by detecting, within predetermined times before and after the peak 84, two peaks 86 and 88 having a predetermined amplitude ratio with respect to the peak 84.

The peak 84 is an example of a “first peak”. The threshold for identifying the peak 84 is an example of a “second threshold”. In addition, the peak 86 is an example of a “second peak”, and the peak 88 is an example of a “third peak”. Therefore, the evaluation unit 26 may identify, in a waveform indicating a temporal change in the distance from the millimeter-wave radar 10 to the neck of the subject 150, the peak 84 indicating a change in distance exceeding the predetermined second threshold.

Next, the evaluation unit 26 may determine whether or not the peak 86, which is a peak within a predetermined amplitude ratio range with respect to the peak 84, has occurred within a predetermined period before the peak 84. The predetermined period used for determination of occurrence of the peak 86 is an example of a “first period”.

Furthermore, the evaluation unit 26 may determine whether or not the peak 88, which is a peak within a predetermined amplitude ratio range with respect to the peak 84, has occurred within a predetermined period after the peak 84. The “first period” and the “second period” may be periods having a same length, or may be periods having different lengths.

The evaluation unit 26 may identify that the three peaks 84, 86, 88 are peaks associated with swallowing, by detecting occurrence of the three peaks 84, 86, 88 within a predetermined period. These three peaks 84, 86, 88 are peaks corresponding to three sounds when the subject 150 performs swallowing. Here, the peak 86 corresponds to a first sound of swallowing, the peak 84 corresponds to a second sound of swallowing, and the peak 88 corresponds to a third sound of swallowing.

Here, the first sound of swallowing corresponds to a closing sound of the epiglottis, the second sound corresponds to a passing sound when food passes through an esophageal entrance, and the third sound corresponds to an opening sound of the epiglottis. Thus, as the peaks of the swallowing sound, the three peaks 84, 86, 88 often appear as a set.

However, in a case where a swallowing disorder occurs, etc., only two peaks of the swallowing sound may appear. In such a case, even when only two peaks (for example, a combination of the peak 84 and the peak 88, or a combination of the peak 84 and the peak 86) appear, there is a case where it may be recognized as a swallowing sound. For example, when amplitudes appear which have magnitudes similar to those of amplitudes of peaks in a case where three peaks of a swallowing sound appear as a set, the evaluation unit 26 may determine that two peaks are peaks corresponding to swallowing. Further, when the amplitude of the peak 84 is equal to or greater than a predetermined threshold (and less than a predetermined different threshold), it may be determined that the peak 84 is a peak corresponding to swallowing.

In this case, the evaluation unit 26 may identify the peak 84 indicating a change in distance exceeding the predetermined second threshold in the waveform indicating the temporal change in distance. Furthermore, the evaluation unit 26 may determine whether or not the peak 86 or the peak 88, which is a peak within the predetermined amplitude ratio range with respect to the peak 84, has occurred within the predetermined period before the peak 84 or within the predetermined period after the peak 84. Further, when the peak 84 and the peak 86 (or the peak 88) have occurred, the evaluation unit 26 may determine that a period including the peak 84 and the peak 86 (or the peak 88) is a period during which the subject 150 is swallowing, and evaluate the swallowing function based on an interval T₂ (or an interval T₃) between the peak 84 and the peak 86 (or the peak 88).

When these peaks associated with swallowing are detected, the evaluation unit 26 evaluates the swallowing function of the subject 150 based on the time interval between the peaks. Further, when the peaks associated with swallowing are detected, the evaluation unit 26 can evaluate whether or not the swallowing function of the subject 150 has declined, by analyzing a waveform pattern of the peaks.

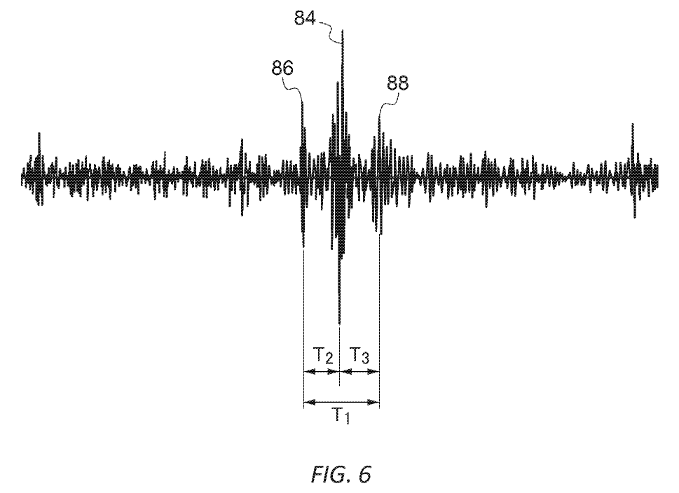

FIG. 6 is an example of the waveform 82 indicating swallowing. For the peaks 84, 86, 88 indicating such swallowing, the evaluation unit 26 can determine presence or absence of the decline in the swallowing function of the subject 150, based on a ratio (interval ratio) of intervals between the peaks 84, 86, 88.

The evaluation unit 26 evaluates the swallowing function based on each time interval of a plurality of peaks 84, 86, 88. When the peak 86 and the peak 88 have occurred, the evaluation unit 26 may determine that a period including the peak 84, the peak 86, and the peak 88 is a period during which the subject 150 is swallowing, and evaluate the swallowing function based on at least one of the interval T₂ between the peak 84 and the peak 86 or the interval T₃ between the peak 84 and the peak 88.

When the swallowing function of the subject 150 has declined, the interval T₃ between the peak 86 corresponding to the second sound and the peak 88 corresponding to the third sound becomes shorter with respect to the interval T₂ between the peak 86 corresponding to the first sound and the peak 84 corresponding to the second sound, with the first sound, the second sound, and the third sound occurring with swallowing.

Therefore, in the evaluation of the swallowing function, when an interval ratio T₂/T₁ of the interval T₂ between the peak 86 and the peak 84 with respect to an interval T₁ between the peak 86 and the peak 88 is equal to or greater than a predetermined threshold, the evaluation unit 26 evaluates that the aspiration risk of the subject 150 is high, and when this interval ratio is smaller than the threshold, the evaluation unit 26 evaluates that the aspiration risk of the subject 150 is low. This threshold is an example of a “third threshold”. When the swallowing function of the subject 150 has declined, the second sound of swallowing approaches the third sound. In swallowing, the evaluation unit 26 evaluates whether or not the second sound is approaching the third sound by comparing the interval ratio T₂/T₁ of the interval T₂ between the first sound and the second sound with respect to the interval T₁ between the first sound and the third sound with the threshold. Here, in the present embodiment, a magnitude of the interval ratio T₂/T₁ is evaluated; however, any ratio may be used as long as it is a ratio by which whether or not the second sound of swallowing is approaching the third sound can be evaluated, and as a ratio for which a threshold is provided, an interval ratio T₃/T₁ may be used, or an interval ratio T₃/T₂ may be used.

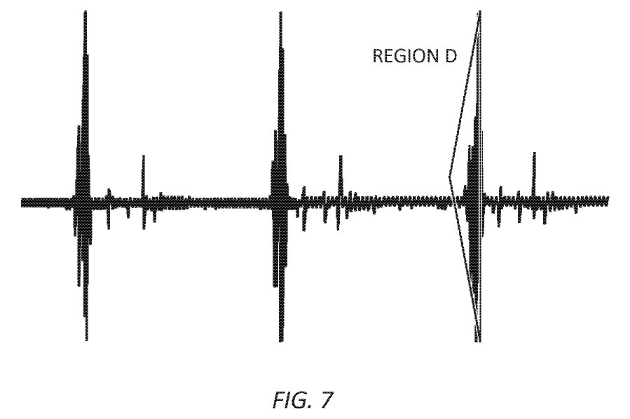

FIG. 7 is an example of a waveform pattern indicating an abnormality in evaluation of presence or absence of an aspiration state. In FIG. 7, similarly to the peaks shown in FIGS. 5 and 6, a plurality of peaks indicating swallowing are shown.

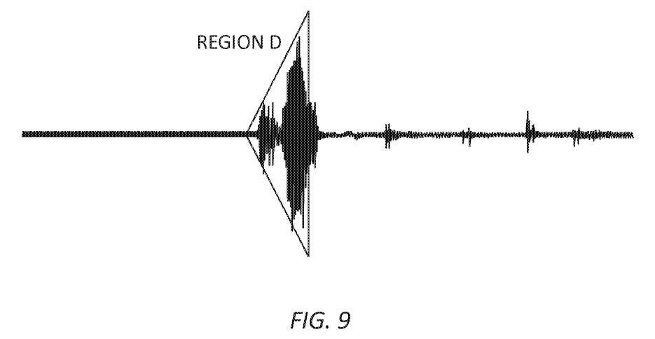

Among the plurality of peaks indicating swallowing, in particular, a waveform pattern shown in region E in the drawing is known to be a waveform pattern in an aspiration state. In FIG. 9, swallowing abnormality will be described by enlarging such a waveform pattern so as to stretch the waveform pattern in a time direction.

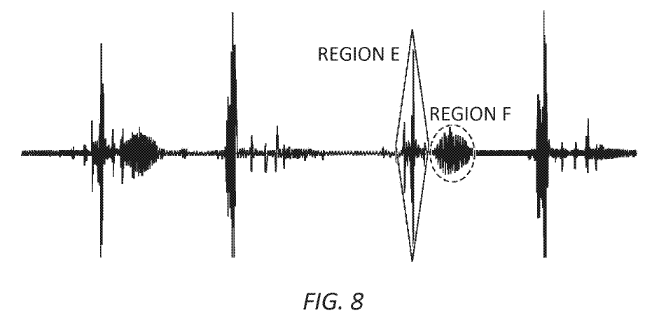

FIG. 8 is an example of a waveform pattern indicating a normal state in the evaluation of presence or absence of an aspiration state. In the waveform pattern of FIG. 8, a waveform pattern of region F appears after region E which is a waveform pattern indicating swallowing.

The pattern of region F is a waveform pattern corresponding to the respiration of the subject 150. After the subject 150 swallows particularly solid or semi-solid food, the waveform pattern corresponding to respiration may appear. In this case, the evaluation unit 26 can evaluate whether or not the subject 150 is in an aspiration state, based on whether or not the waveform pattern corresponding to respiration is a clear dry respiration sound.

FIG. 9 is an example of the enlarged view obtained by stretching FIG. 7 in the time direction around region D. Region D has a leftward-convex triangular shape. In a normal pattern of a swallowing state, since three peaks appear, a region surrounding an outline of the peaks exhibits a substantially rhombic shape.

The evaluation unit 26 may determine whether or not the subject 150 is in an aspiration state, based on whether the rhombic waveform pattern is generated or whether or not the pattern is close to a leftward-convex triangular shape. In this manner, the evaluation unit 26 may identify the peaks 84, 86, 88 of changes in distance exceeding the predetermined threshold in the waveform indicating the temporal change in distance. Furthermore, the evaluation unit 26 may evaluate that the subject 150 is in an aspiration state, when a waveform within a predetermined time including the peaks 84, 86, 88 satisfies a predetermined condition indicating aspiration, such as that a region surrounding an outline of the peaks has a leftward-convex triangular shape.

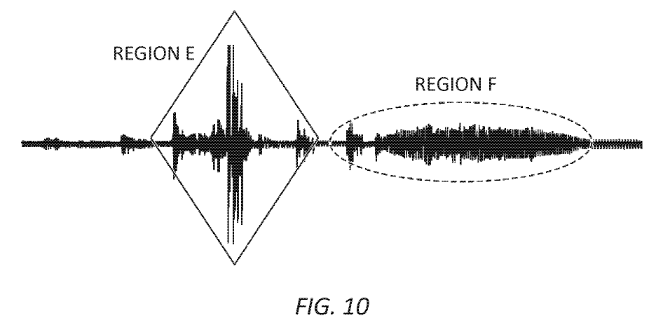

FIG. 10 is an example of an enlarged view obtained by stretching FIG. 8 around region E and region F. In region E, a waveform pattern surrounding a series of peaks indicating swallowing indicates a rhombic shape. In a waveform pattern indicating normal swallowing, a series of waveform patterns surrounding peaks indicating swallowing indicates a rhombus.

Furthermore, after eating semi-solid food, a signal corresponding to a clear dry respiration sound as shown in region F appears. On the other hand, when such a signal corresponding to a respiration sound does not appear after eating semi-solid food, a food bolus remains in the pharynx after swallowing, and aspiration is likely to occur immediately after swallowing. In addition, when the subject 150 is in an aspiration state, coughing may occur immediately after swallowing.

Further, when the subject 150 is in an aspiration state, as a sound of swallowing itself, a choking sound, a weak sound, a reflux sound, a reflex delayed sound, a continuous sound, or the like may appear, and a signal corresponding to such a sound may appear. Furthermore, when the subject 150 is in an aspiration state, as a respiration sound after swallowing, a sound corresponding to choking (expectorated sound), a wet expiratory sound, wet hoarseness, or the like may occur. Therefore, the evaluation unit 26 can determine whether or not the subject 150 is in an aspiration state, based on the temporal change in distance from millimeter-wave radar 10 to the neck (such as vibration or displacement of the neck) corresponding to the swallowing sound and the respiration sound after swallowing.

In this manner, the evaluation unit 26 may identify a temporal change in distance corresponding to the respiration of the subject 150, which occurs within a predetermined period from the peak corresponding to swallowing. The predetermined period for identifying a temporal change in the distance corresponding to this respiration is an example of a “third period”. When the temporal change in the distance corresponding to the respiration of the subject 150 satisfies a predetermined condition, the evaluation unit 26 evaluates that the subject 150 is in an aspiration state. The predetermined condition is, for example, whether or not there is a cough, and whether or not the respiration sound after swallowing is determined as a sound corresponding to the above-described choking (expectorated sound), a wet expiratory sound, wet hoarseness, or the like. In particular, regarding whether or not there is a “cough”, the evaluation unit 26 may evaluate the aspiration state of the subject 150, further based on a detection result of a temporal change in the distance corresponding to coughing of the subject 150 occurring within a predetermined period.

In this manner, the evaluation unit 26 may evaluate whether or not the subject 150 is in an aspiration state, based on the temporal change in the distance corresponding to the respiration sound after swallowing, in addition to the temporal change in the distance corresponding to the sound of swallowing itself. When the subject 150 is evaluated by the evaluation unit 26 as being in an aspiration state, the notification unit 42 may issue an alert.

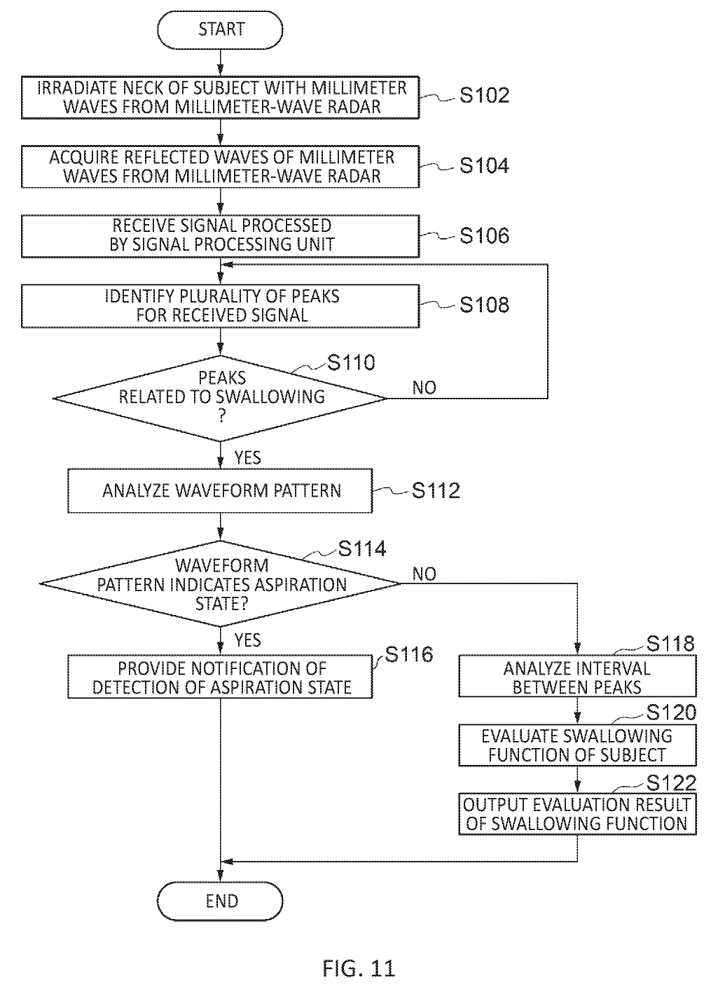

FIG. 11 is an example of a flowchart for describing an evaluation method of an aspiration state and a swallowing function according to the embodiment. The evaluation method includes steps S102 to S122.

The evaluation system 100 irradiates the neck of the subject 150 with millimeter waves from the millimeter-wave radar 10 (S102). The evaluation system 100 may control a direction in which the millimeter waves are radiated by the radar control unit 28.

The signal acquisition unit 22 acquires reflected waves of the millimeter waves from the millimeter-wave radar (S104). As described with reference to FIG. 4, the signal acquisition unit 22 may AD-convert an analog signal and output a signal of the reflected waves as a digital signal.

The evaluation unit 26 receives the signal processed by the signal processing unit 24 (S106). The correction unit 32 may correct signal extraction by the signal processing unit 24, based on posture information acquired by the posture acquisition unit 30.

The evaluation unit 26 identifies a plurality of peaks in the received signal (S108). Identification of the plurality of peaks performed by the evaluation unit 26 may be based on a threshold provided for an amplitude. Thus, the evaluation unit 26 identifies vibration of the neck based on swallowing.

The evaluation unit 26 determines whether or not the peaks identified in S108 are peaks related to swallowing of the subject 150 (S110). If the peaks are peaks related to swallowing, the process proceeds to S112, and if the peaks are not peaks related to swallowing, the process returns to S108 to identify the peaks related to swallowing.

If the peaks are peaks related to swallowing, the evaluation unit 26 evaluates whether or not the subject 150 is in an aspiration state. As in the present embodiment, evaluation by the evaluation unit 26 of whether or not the subject 150 is in an aspiration state may be performed prior to evaluation of the swallowing function of the subject 150 in order to determine whether or not the subject 150 is in a dangerous state at that time. However, in the evaluation system 100, only one of the evaluation of the aspiration state and the evaluation of the swallowing function may be implemented, and even when both are executed, they may be executed in parallel. Further, an order in which the evaluation is performed is not limited to the present embodiment.

The evaluation unit 26 analyzes a waveform pattern in a signal indicating vibration of the neck of the subject 150 or the like in order to evaluate whether or not the subject 150 is in an aspiration state (S112). Analysis of the waveform pattern by the evaluation unit 26 may be performed based on a shape of a region surrounding the peaks indicating swallowing.

It is determined whether or not the waveform pattern indicates an aspiration state (S114). The evaluation unit 26 may evaluate that the subject 150 is not in an aspiration state when the shape of the region surrounding the peaks indicating swallowing is a substantially rhombic shape, and may evaluate that the subject 150 is in an aspiration state when the shape of the region surrounding the peaks indicating swallowing is a leftward-convex triangular shape. Furthermore, as evaluation of the waveform pattern, the evaluation unit 26 may evaluate whether or not the subject 150 is in an aspiration state, based on a predetermined condition at a peak corresponding to a respiration sound that follows after the peak indicating swallowing. If an evaluation result regarding presence or absence of an aspiration state by the evaluation unit 26 indicates the aspiration state, the process proceeds to step S116, and if the evaluation result regarding presence or absence of an aspiration state by the evaluation unit 26 indicates a non-aspiration state, the process proceeds to S118.

If the evaluation result by the evaluation unit 26 indicates that the subject 150 is in an aspiration state, the notification unit 42 provides notification of detection of an aspiration state (S116). This notification may be an alert that the subject 150 is in an aspiration state. In this case, the display unit 40 may also display that the subject 150 is in an aspiration state.

If the evaluation result by the evaluation unit 26 indicates a non-aspiration state, the evaluation unit 26 may evaluate the swallowing function of the subject 150. For this purpose, the evaluation unit 26 analyzes an interval between peaks (S118).

The evaluation unit 26 evaluates the swallowing function of the subject (S120). Evaluation of the swallowing function of the subject 150 by the evaluation unit 26 may be performed based on a ratio of intervals between peaks corresponding to swallowing sounds. In the evaluation of the swallowing function, when the ratio T₂/T₁ of the interval T₂ between the peak 86 and the peak 84 to the interval T₁ between the peak 86 and the peak 88 is equal to or greater than a predetermined threshold, the evaluation unit 26 evaluates that an aspiration risk of the subject 150 is high, and when this ratio is smaller than the threshold, the evaluation unit 26 may evaluate that the aspiration risk of the subject 150 is low.

The evaluation result of the swallowing function is output (S122). Output of the evaluation result may be performed via the display unit 40 and the notification unit 42, and may be performed visually or may be performed by voice. In addition, the output of the evaluation result may be performed to a medical system connected via the network 160, and may be linked with a meal provision schedule or a swallowing training schedule of the subject 150, or the like.

Through the above processing, in the evaluation method of the present embodiment, the swallowing function and the aspiration state of the subject 150 can be evaluated by a non-contact and low-noise method, based on vibration and displacement, or the like, of the neck accompanying swallowing of the subject 150, by using millimeter waves. Accordingly, the swallowing function and the aspiration state of the subject 150 can be evaluated with a small burden on the subject 150 and with high accuracy.

FIG. 12 is a flowchart illustrating an example of processing for posture information generation performed by the posture acquisition unit 30. The processing for the posture information generation includes steps S202 to S218.

The posture acquisition unit 30 acquires, from the posture detection unit 14, a signal corresponding to a posture of the subject 150 (S202). The posture detection unit 14 may detect the posture of the subject 150 by millimeter waves, and may detect the posture of the subject 150 by LiDAR. In addition, the posture detection unit 14 may include a camera, and the posture acquisition unit 30 may output posture information of the subject 150 based on an image or video captured by the camera.

The posture acquisition unit 30 performs preprocessing on the signal detected by the posture detection unit 14 (S204). Specific examples of the preprocessing performed here include calibration such as noise removal from the acquired signal or correction of deviations between sensors and between environments in the posture detection unit 14.

The posture acquisition unit 30 performs spatiotemporal synchronization on the preprocessed data, and unifies coordinate systems of data output from sensors into a global coordinate system (S206). With respect to a local coordinate system of each sensor, a difference in data collection time for each sensor is adjusted and a difference in positional relationship between sensors is corrected using a calibration matrix.

The posture acquisition unit 30 performs distance, phase, and frequency analysis on the data acquired in S206 (S208). Specifically, the distance may be calculated based on Time of Flight (TOF) until a transmitted signal is reflected and returns, and when an FMCW-type radar is used, distance FFT may be performed and the distance may be calculated from a peak position.

Next, the posture acquisition unit 30 may estimate an angle of a target by beamforming or an angle FFT, etc. (S210). By S208 and S210, a distance and an angle of a point cloud of each measurement target point are identified, and 3D point cloud data can be generated.

The posture acquisition unit 30 transforms a position of the point cloud into a three-dimensional coordinate system, based on the identified distance and angle, and generates a point cloud in the three-dimensional space (S212). Furthermore, the posture acquisition unit 30 detects a human body region and separates (segments) points corresponding to the human body from the generated point cloud to extract a point cloud corresponding to the person (S214).

After the human body region can be identified, the posture acquisition unit 30 estimates regions corresponding to the upper body and the head (S216). For the point cloud of the human body, clustering is performed on a collection of spatially close points, etc., and labeling is performed on parts by using machine learning, etc., to classify the parts. Thus, the parts of the human body are subdivided.

Thus, the posture acquisition unit 30 becomes capable of calculating, in 3D coordinates, the target point in the point cloud of the head, and becomes capable of tracking a temporal change (S218). Thus, the posture acquisition unit 30 becomes capable of estimating a head position, and becomes capable of outputting the posture information based on the head position of the subject 150. As described above, the posture information based on the head position of the subject 150 is generated.

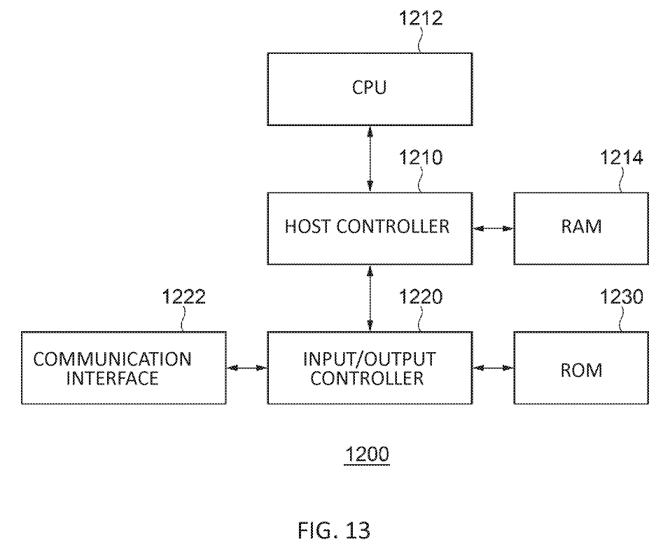

FIG. 13 is a diagram illustrating an example of a computer 1200 that can embody, in whole or in part, aspects of the present embodiment. A program installed in the computer 1200 can cause the computer 1200 to function as operations associated with the apparatus according to the embodiment of the present invention or as one or more “units” of the apparatus. Alternatively, the program can cause the computer 1200 to execute the operations or the one or more “units”. The program can cause the computer 1200 to execute the process according to the embodiment of the present invention or steps of the process. Such a program may be executed by a CPU 1212 to cause the computer 1200 to execute specific operations associated with some or all of blocks of the flowcharts and block diagrams described in the present specification.

The computer 1200 according to the present embodiment includes the CPU 1212 and a RAM 1214, and they are interconnected by the host controller 1210. The computer 1200 also includes a communication interface 1222 and an input/output unit, and they are connected to a host controller 1210 via an input/output controller 1220. The computer 1200 also includes a ROM 1230. The CPU 1212 operates in accordance with programs stored in the ROM 1230 and the RAM 1214, thereby controlling each unit.

The communication interface 1222 communicates with other electronic devices via the network. A hard disk drive may store programs and data used by the CPU 1212 in the computer 1200. The ROM 1230 stores therein a boot program, etc., executed by the computer 1200 upon activation, and/or a program dependent on hardware of the computer 1200. The program is provided via a computer-readable recording medium such as a CR-ROM, a USB memory, or an IC card, or via the network. The program is installed in the RAM 1214, which is also an example of the computer-readable recording medium, or in the ROM 1230, and is executed by the CPU 1212. Information processing described in these programs is read by the computer 1200, and brings about cooperation between the programs and various types of hardware resources described above. The apparatus or the method may be configured by realizing the operation or processing of information in accordance with use of the computer 1200.

For example, when communication is performed between the computer 1200 and an external device, the CPU 1212 may execute a communication program loaded into the RAM 1214, and may instruct the communication interface 1222 to perform communication processing based on processing described in the communication program. The communication interface 1222, under control of the CPU 1212, reads transmission data stored in the transmission buffer area provided in the RAM 1214 or in a recording medium such as a USB memory, transmits the read transmission data to the network, or writes reception data received from the network into the reception buffer area or the like provided on the recording medium.

In addition, the CPU 1212 may cause all or a necessary part of the file or the database stored in an external recording medium such as a USB memory or the like to be read into the RAM 1214, and may execute various types of processing on data on the RAM 1214. The CPU 1212 may then write back the processed data to the external recording medium.

Various types of information such as various types of programs, data, tables, and databases are stored in the recording medium and may be subjected to information processing. The CPU 1212 may execute, on data read from the RAM 1214, various types of processing including various types of operations, information processing, conditional determination, conditional branching, unconditional branching, retrieval/replacement of information, and the like, which are described throughout the present disclosure and specified by the instruction sequence of the program, and writes back results to the RAM 1214. In addition, the CPU 1212 may retrieve information in the file, the database, or the like in the recording medium. For example, when a plurality of entries each having an attribute value of a first attribute associated with an attribute value of a second attribute are stored in a recording medium, the CPU 1212 may search, from among the plurality of entries, for the entry that matches the condition in which the attribute value of the first attribute is specified, read the attribute value of the second attribute stored in the entry, and thereby obtain the attribute value of the second attribute associated with the first attribute that satisfies a predetermined condition.

The program or software module described above may be stored in the computer-readable storage medium on the computer 1200 or in the vicinity of the computer 1200. In addition, the recording medium such as a hard disk or RAM provided in the server system connected to a dedicated communication network or the Internet can be used as a computer-readable storage medium, thereby providing a program to the computer 1200 via the network.

A computer-readable medium may include any tangible device capable of storing instructions to be executed by the appropriate device. As a result, the computer-readable medium having instructions stored therein constitutes a product including instructions that may be executed to create means for executing operations specified in the flowchart or the block diagram. Examples of the computer-readable medium may include an electronic storage medium, a magnetic storage medium, an optical storage medium, an electromagnetic storage medium, a semiconductor storage medium, and the like. More specific examples of the computer-readable medium may include a FLOPPY (registered trademark) disk, a diskette, a hard disk, a random access memory (RAM), a read-only memory (ROM), an erasable programmable read-only memory (EPROM or flash memory), an electrically erasable programmable read-only memory (EEPROM (registered trademark)), a static random access memory (SRAM), a compact disc read-only memory (CD-ROM), a digital versatile disc (DVD), a BLU-RAY (RTM) disc, a memory stick, an integrated circuit card, and the like.

A computer-readable instruction may include either source code or object code written in any combination of one or more programming languages. The source code or the object code includes a conventional procedural programming language. The conventional procedural programming language may be an assembler instruction, an instruction set architecture (ISA) instruction, a machine instruction, a machine-dependent instruction, a microcode, a firmware instruction, state-setting data, or an object-oriented programming language such as SMALLTALK (registered trademark), JAVA (registered trademark), C++, and the like, and may be a “C” programming language or a similar programming language. The computer-readable instruction may be provided to the processor or a programmable circuit of a general-purpose computer, a special-purpose computer, or another programmable data processing apparatus, locally or via the local area network (LAN), a wide area network (WAN) such as the Internet, or the like. The processor or the programmable circuit may execute the computer-readable instruction to create means for executing operations specified in the flowchart or the block diagram. Examples of the processor include a computer processor, a processing unit, a microprocessor, a digital signal processor, a controller, a microcontroller, and the like.

Although description has been given above by way of the embodiments, the technical scope of the present invention is not limited to in the above-described embodiments. It is apparent to persons skilled in the art that various alterations or improvements can be added to the above-described embodiments. It is also apparent from the scope of the claims that the embodiments added with such alterations or improvements can be included in the technical scope of the invention.

The operations, procedures, steps, and stages of each process performed by an apparatus, system, program, and method shown in the claims, embodiments, or diagrams can be performed in any order as long as the order is not indicated by "prior to," "before," or the like and as long as the output from a previous process is not used in a later process. Even if the process flow is described using phrases such as "first" or "next" in the claims, embodiments, or diagrams, it does not necessarily mean that the process must be performed in this order.

### OTHER POSSIBLE ITEMS

### Item 1

An evaluation apparatus including: a signal acquisition unit which acquires a signal indicating a temporal change in a distance from a millimeter-wave radar to a body surface of a subject, the signal being obtained by the millimeter-wave radar receiving reflected waves of millimeter waves radiated from the millimeter-wave radar to the body surface; and an evaluation unit which evaluates at least one of a swallowing function or an aspiration state of the subject based on the temporal change in the distance indicated by the signal.

### Item 2

The evaluation apparatus according to item 1, in which the body surface is a surface of a neck of the subject.

### Item 3

The evaluation apparatus according to item 1, in which the evaluation unit identifies a peak in a waveform indicating the temporal change in the distance included within a predetermined period by statistically analyzing the waveform, and the evaluation unit evaluates the swallowing function by a swallowing function index that is a numerical value to which a higher number is assigned when the swallowing function is at a high level and to which a lower numerical value is assigned when the swallowing function is at a low level.

### Item 4

The evaluation apparatus according to item 1, in which the evaluation unit identifies a plurality of peaks indicating changes in distance exceeding a predetermined first threshold, in a waveform indicating the temporal change in the distance included within a predetermined period, and the evaluation unit evaluates the swallowing function based on each time interval of the plurality of peaks.

### Item 5

The evaluation apparatus according to item 4, in which the evaluation unit identifies a first peak indicating a change in distance exceeding a predetermined second threshold in a waveform indicating the temporal change in the distance, the evaluation unit determines whether or not a second peak, which is a peak within a predetermined amplitude ratio range with respect to the first peak, occurs within a predetermined first period before the first peak or within a predetermined period after the first peak, and the evaluation unit, when the first peak and the second peak occur, determines that a period including the first peak and the second peak is a period during which the subject is swallowing, and evaluates the swallowing function based on a period between the first peak and the second peak.

### Item 6

The evaluation apparatus according to item 4, in which the evaluation unit identifies a first peak indicating a change in distance exceeding a predetermined second threshold, in a waveform indicating the temporal change in the distance, the evaluation unit determines whether or not a second peak, which is a peak within a predetermined amplitude ratio range with respect to the first peak, occurs within a predetermined first period before the first peak, the evaluation unit determines whether or not a third peak, which is a peak within a predetermined amplitude ratio range with respect to the first peak, occurs within a predetermined second period after the first peak, and the evaluation unit, when the second peak and the third peak occur, determines that a period including the first peak, the second peak, and the third peak is a period during which the subject is swallowing, and evaluates the swallowing function based on at least one of an interval between the second peak and the first peak or an interval between the first peak and the third peak.

### Item 7

The evaluation apparatus according to item 6, in which, in evaluation of the swallowing function, the evaluation unit evaluates that an aspiration risk of the subject is high when an interval ratio of an interval between the second peak and the first peak to the interval between the second peak and the third peak is equal to or greater than a predetermined third threshold, and evaluates that the aspiration risk of the subject is low when the interval ratio is smaller than the predetermined third threshold.

### Item 8

The evaluation apparatus according to item 1, in which the evaluation unit identifies a peak of a change in distance exceeding a predetermined threshold, in a waveform indicating the temporal change in the distance, and the evaluation unit evaluates that the subject is in an aspiration state, when a waveform within a predetermined time including the peak satisfies a predetermined condition indicating aspiration.

### Item 9

The evaluation apparatus according to item 8, in which the evaluation unit identifies a peak of a change in distance exceeding a predetermined first threshold in the waveform indicating the temporal change in the distance, thereby identifying a peak, corresponding to swallowing, of the change in the distance,

identifies the temporal change in the distance corresponding to respiration of the subject occurring within a predetermined third period from the peak corresponding to the swallowing, and evaluates that the subject is in an aspiration state, when the temporal change in the distance corresponding to respiration of the subject satisfies a predetermined condition.

### Item 10

The evaluation apparatus according to item 9, in which the evaluation unit evaluates the aspiration state of the subject further based on a detection result of the temporal change in the distance corresponding to coughing of the subject occurring within the predetermined third period.

### Item 11

The evaluation apparatus according to item 1, further including: a direction detection unit which detects a direction of the body surface with respect to the millimeter-wave radar for radiating the millimeter waves; and a radar control unit which outputs, to the millimeter-wave radar, a control signal for controlling, based on the direction of the body surface detected by the direction detection unit, a direction of the millimeter waves output from the millimeter-wave radar, such that the millimeter wave radiated from the millimeter-wave radar is radiated to the body surface.

### Item 12

The evaluation apparatus according to item 11, in which the direction detection unit detects, as the direction of the body surface, a direction of a vicinity of a point, which is most recessed in a viewpoint as viewed from the millimeter-wave radar, on the body surface on an occipital side from a head to a torso of the subject.

### Item 13

The evaluation apparatus according to item 11, in which the direction detection unit detects, as a direction of a neck, a direction in a vicinity of a point, which is most recessed in a viewpoint as viewed from the millimeter-wave radar, on the body surface on an occipital side from a head to a torso of the subject, and estimates a direction of the head or the torso of the subject as the direction of the body surface by using distance information from the millimeter-wave radar to the neck.

### Item 14

The evaluation apparatus according to item 11, in which the direction detection unit detects, as the direction of the body surface, a direction from the millimeter-wave radar toward a center point in a width of a portion, in which the width is narrowest, on the body surface, the width being defined in a direction intersecting a direction from a head to a torso of the subject.

### Item 15

The evaluation apparatus according to item 1, in which the evaluation unit estimates at least one of the swallowing function or the aspiration state of the subject with respect to the temporal change in the distance indicated by the signal detected by the signal acquisition unit by using a machine learning model that has been machine-trained with a temporal change in a distance from the millimeter-wave radar to a body surface of a subject as an input and at least one of the swallowing function or the aspiration state as an output, thereby evaluating at least one of the swallowing function or the aspiration state of the subject.

### Item 16

The evaluation apparatus according to item 1, including a biological information acquisition unit which acquires biological information regarding a living body of the subject including predetermined items affecting the swallowing function of the subject, in which the evaluation unit evaluates at least one of the swallowing function or the aspiration state of the subject further based on the biological information.

### Item 17

The evaluation apparatus according to item 16, in which the biological information includes a medical record including predetermined items affecting the swallowing function of the subject.

### Item 18

The evaluation apparatus according to item 1, including a schedule acquisition unit which acquires a schedule of at least one of a mealtime provided to the subject or a sleep time of the subject, in which the evaluation unit evaluates at least one of the swallowing function or the aspiration state of the subject further based on the schedule of at least one of the mealtime or the sleep time.

### Item 19

The evaluation apparatus according to item 1, including a chewing count acquisition unit which acquires a chewing count detected by a chewing count detection unit, in which the evaluation unit evaluates at least one of the swallowing function or the aspiration state of the subject further based on the chewing count.

### Item 20

The evaluation apparatus according to item 1, including a posture acquisition unit which acquires posture information of the subject from a posture detection unit which detects a posture of the subject, in which the evaluation unit evaluates at least one of the swallowing function or the aspiration state of the subject further based on the posture information.

### Item 21

The evaluation apparatus according to item 1, further including a notification unit which provides notification of an evaluation result by the evaluation unit.

### Item 22

An evaluation system including: the evaluation apparatus according to any one of items 1 to 21; and the millimeter-wave radar.

### Item 23

An evaluation system installation member including: the evaluation system according to item 22; and a support portion which supports the millimeter-wave radar.

### Item 24

A chair comprising: the evaluation system according to item 22, a seat surface for the subject to sit on; and a back surface which extends from one side of the seat surface and has a support portion which supports the millimeter-wave radar such that the millimeter-wave radar is located within a predetermined range from the neck of the subject.

### Item 25

A bed including: the evaluation system according to item 22; a bed base for the subject to lie on; a headboard which extends from one side of the bed base that is on a head side of the subject when the subject lies down; and a support portion which is provided on either one of the bed base or the headboard and supports the millimeter-wave radar such that the millimeter-wave radar is located within a predetermined range from the neck of the subject.

### Item 26

An evaluation method including: acquiring a signal indicating a temporal change in a distance from a millimeter-wave radar to a body surface of a subject, the signal being obtained by receiving reflected waves of millimeter waves radiated from the millimeter-wave radar to the body surface; and evaluating at least one of a swallowing function or an aspiration state of the subject based on the detection result of the temporal change in the distance.

### Item 27

The evaluation method according to item 26, in which the body surface is a surface of a neck of the subject.

### Item 28

A program that, when executed by a computer, causes the computer to perform operations including: acquiring a signal indicating a temporal change in a distance from a millimeter-wave radar to a body surface of a subject, the signal being obtained by receiving reflected waves of millimeter waves radiated from the millimeter-wave radar to the body surface, and evaluating at least one of a swallowing function or an aspiration state of the subject based on a detection result of the temporal change in the distance.

### Item 29

The program according to item 28, in which the body surface is a surface of a neck of the subject.

### EXPLANATION OF REFERENCES

10: millimeter-wave radar;
12: direction detection unit;
14: posture detection unit;
16: chewing count detection unit;
20: control unit;
22: signal acquisition unit;
24: signal processing unit;
26: evaluation unit;
28: radar control unit;
30: posture acquisition unit;
32: correction unit;
34: biological information acquisition unit;
36: schedule acquisition unit;
38: chewing count acquisition unit;
40: display unit;
42: notification unit;
50: RFIC;
52: AC power supply;
54: amplifier;
60: level detection unit;
62: distance FFT unit;
64: beamforming unit;
68: tan⁻¹ demodulation unit;
70: bandpass filter;
72: multiplier;
80, 82: waveform;
84, 86, 88: peak;
100: evaluation system;
102: transmitting antenna;
104: receiving antenna;
150: subject;
152: user terminal;
160: network;
162: server;
164: monitor;
200: bed;
210: bed base;
220: headboard;
222: amplifier;
224: multiplier;
226: low-pass filter;
228: ADC;
230: bedding;
300: chair;
310: seat surface;
320: back surface;
322: distance range selection unit;
324: direction range selection unit; and
330: support portion.

What is claimed is:

1. An evaluation apparatus comprising:
a signal acquisition unit which acquires a signal indicating a temporal change in a distance from a millimeter-wave radar to a body surface of a subject, the signal being obtained by the millimeter-wave radar receiving reflected waves of millimeter waves radiated from the millimeter-wave radar to the body surface; and
an evaluation unit which evaluates at least one of a swallowing function or an aspiration state of the subject based on the temporal change in the distance indicated by the signal.

2. The evaluation apparatus according to claim 1, wherein the body surface is a surface of a neck of the subject.

3. The evaluation apparatus according to claim 1, wherein
the evaluation unit identifies a peak in a waveform indicating the temporal change in the distance included within a predetermined period by statistically analyzing the waveform, and
the evaluation unit evaluates the swallowing function by a swallowing function index that is a numerical value to which a higher number is assigned when the swallowing function is at a high level and to which a lower numerical value is assigned when the swallowing function is at a low level.

4. The evaluation apparatus according to claim 1, wherein
the evaluation unit identifies a plurality of peaks indicating changes in distance exceeding a predetermined first threshold, in a waveform indicating the temporal change in the distance included within a predetermined period, and
the evaluation unit evaluates the swallowing function based on each time interval of the plurality of peaks.

5. The evaluation apparatus according to claim 4, wherein
the evaluation unit identifies a first peak indicating a change in distance exceeding a predetermined second threshold in a waveform indicating the temporal change in the distance,
the evaluation unit determines whether or not a second peak, which is a peak within a predetermined amplitude ratio range with respect to the first peak, occurs within a predetermined first period before the first peak or within a predetermined period after the first peak, and
the evaluation unit, when the first peak and the second peak occur, determines that a period including the first peak and the second peak is a period during which the subject is swallowing, and evaluates the swallowing function based on a period between the first peak and the second peak.

6. The evaluation apparatus according to claim 4, wherein
the evaluation unit identifies a first peak indicating a change in distance exceeding a predetermined second threshold, in a waveform indicating the temporal change in the distance,
the evaluation unit determines whether or not a second peak, which is a peak within a predetermined amplitude ratio range with respect to the first peak, occurs within a predetermined first period before the first peak,
the evaluation unit determines whether or not a third peak, which is a peak within a predetermined amplitude ratio range with respect to the first peak, occurs within a predetermined second period after the first peak, and
the evaluation unit, when the second peak and the third peak occur, determines that a period including the first peak, the second peak, and the third peak is a period during which the subject is swallowing, and evaluates the swallowing function based on at least one of an interval between the second peak and the first peak or an interval between the first peak and the third peak.

7. The evaluation apparatus according to claim 6, wherein, in evaluation of the swallowing function, the evaluation unit evaluates that an aspiration risk of the subject is high when an interval ratio of the interval between the second peak and the first peak to the interval between the second peak and the third peak is equal to or greater than a predetermined third threshold, and evaluates that the aspiration risk of the subject is low when the interval ratio is smaller than the third threshold.

8. The evaluation apparatus according to claim 1, wherein
the evaluation unit identifies a peak of a change in distance exceeding a predetermined threshold, in a waveform indicating the temporal change in the distance, and
the evaluation unit evaluates that the subject is in an aspiration state, when a waveform within a predetermined time including the peak satisfies a predetermined condition indicating aspiration.

9. The evaluation apparatus according to claim 8, wherein
the evaluation unit identifies a peak of a change in distance exceeding a predetermined first threshold in the waveform indicating the temporal change in the distance, thereby identifying a peak, corresponding to swallowing, of the change in distance,
identifies the temporal change in the distance corresponding to respiration of the subject occurring within a predetermined third period from the peak corresponding to swallowing, and
evaluates that the subject is in an aspiration state, when the temporal change in the distance corresponding to respiration of the subject satisfies a predetermined condition.

10. The evaluation apparatus according to claim 9, wherein the evaluation unit evaluates the aspiration state of the subject further based on a detection result of the temporal change in the distance corresponding to coughing of the subject occurring within the predetermined third period.

11. The evaluation apparatus according to claim 1, further comprising:
a direction detection unit which detects a direction of the body surface with respect to the millimeter-wave radar for radiating the millimeter waves; and
a radar control unit which outputs, to the millimeter-wave radar, a control signal for controlling, based on the direction of the body surface detected by the direction detection unit, a direction of the millimeter waves output from the millimeter-wave radar, such that the millimeter wave radiated from the millimeter-wave radar is radiated to the body surface.

12. The evaluation apparatus according to claim 11, wherein the direction detection unit detects, as the direction of the body surface, a direction of a vicinity of a point, which is most recessed in a viewpoint as viewed from the millimeter-wave radar, on the body surface on an occipital side from a head to a torso of the subject.

13. The evaluation apparatus according to claim 11, wherein the direction detection unit detects, as a direction of a neck, a direction of a vicinity of a point, which is most recessed in a viewpoint as viewed from the millimeter-wave radar, on the body surface on an occipital side from a head to a torso of the subject, and estimates a direction of the head or the torso of the subject as the direction of the body surface by using distance information from the millimeter-wave radar to the neck.

14. The evaluation apparatus according to claim 11, wherein the direction detection unit detects, as the direction of the body surface, a direction from the millimeter-wave radar toward a center point in a width of a portion, in which the width is narrowest, on the body surface, the width being defined in a direction intersecting a direction from a head to a torso of the subject.

15. The evaluation apparatus according to claim 1, wherein the evaluation unit estimates at least one of the swallowing function or the aspiration state of the subject with respect to the temporal change in the distance indicated by the signal detected by the signal acquisition unit by using a machine learning model that has been machine-trained with a temporal change in a distance from the millimeter-wave radar to a body surface of a subject as an input and at least one of the swallowing function or the aspiration state as an output, thereby evaluating at least one of the swallowing function or the aspiration state of the subject.

16. The evaluation apparatus according to claim 1, comprising
a biological information acquisition unit which acquires biological information regarding a living body of the subject including predetermined items affecting the swallowing function of the subject, wherein
the evaluation unit evaluates at least one of the swallowing function or the aspiration state of the subject further based on the biological information.

17. The evaluation apparatus according to claim 16, wherein the biological information includes a medical record including predetermined items affecting the swallowing function of the subject.

18. The evaluation apparatus according to claim 1, comprising
a schedule acquisition unit which acquires a schedule of at least one of a mealtime provided to the subject or a sleep time of the subject, wherein
the evaluation unit evaluates at least one of the swallowing function or the aspiration state of the subject further based on the schedule of at least one of the mealtime or the sleep time.

19. The evaluation apparatus according to claim 1, comprising
a chewing count acquisition unit which acquires a chewing count detected by a chewing count detection unit, wherein
the evaluation unit evaluates at least one of the swallowing function or the aspiration state of the subject further based on the chewing count.

20. The evaluation apparatus according to claim 1, comprising
a posture acquisition unit which acquires posture information of the subject from a posture detection unit which detects a posture of the subject, wherein
the evaluation unit evaluates at least one of the swallowing function or the aspiration state of the subject further based on the posture information.
